(19) United States
(12) Patent Application Publication
(10) Pub. No.: US 2025/0381014 A1
(43) Pub. Date: Dec. 18, 2025

(54) CUSTOMIZABLE DENTAL PROSTHESIS FOR USE WITH DIGITAL WORKFLOWS

(71) Applicant: BIOMET 3I, LLC, Palm Beach Gardens, FL (US)

(72) Inventors: Zachary B. Suttin, Jupiter, FL (US); Andreas Geier, Gargazon (IT); Harald Egger, Lana (IT)

(21) Appl. No.: 19/236,754

(22) Filed: Jun. 12, 2025

Related U.S. Application Data
(60) Provisional application No. 63/659,128, filed on Jun. 12, 2024.

(51) Int. Cl. A61C 8/00; A61C 13/34; G06T 17/00; G06T 19/20; G16H 10/60

(52) U.S. Cl. CPC A61C 8/0089; A61C 8/008; A61C 13/34; G06T 17/00; G06T 19/20; G16H 10/60; G06T 2210/41; G06T 2219/2004

(57) ABSTRACT

The present disclosure is directed to a computational system and method that receive fixture scan data comprising an image of a scan fixture engaging a scan fixture calibration object and a dental analog having a nonrotational structure orientation, generate, from the fixture scan data, a virtual three-dimensional model of the scan fixture and scan fixture calibration object, and replace, in the virtual attachment member three-dimensional model, the image of the scan fixture with a virtual counterpart of the scan fixture calibration object to form a modified virtual three-dimensional fixture model comprising one or more of a reference coordinate system position relative to a surface of the scan fixture and an orientation of the nonrotational structure relative to a surface of the scan fixture.

### CROSS REFERENCE TO RELATED APPLICATION

The present application claims the benefits of U.S. Provisional Application Ser. No. 63/659,128, filed Jun. 12, 2024, entitled “Customizable Dental Prosthesis for Use in Digital Workflows”, which is incorporated herein by this reference in its entirety.

### FIELD

The disclosure relates generally to digital dental workflows and particularly to digital dental restoration workflows.

### BACKGROUND OF THE INVENTION

The dental restoration of a partially or wholly edentulous patient with artificial dentition is typically done in two stages. In the first stage, an incision is made through the gingiva to expose the underlying bone. An artificial tooth root, in the form of a dental implant, is placed in the jawbone for osseointegration. The dental implant generally includes a threaded bore to receive a retaining screw for holding mating components thereon. During the first stage, the gum tissue overlying the implant is sutured and heals as the osseointegration process continues.

Once the osseointegration process is complete, the second stage is initiated. Here, the gingival tissue is re-opened to expose an end of the dental implant. A healing component or healing abutment is fastened to the exposed end of the dental implant to allow the gingival tissue to heal therearound. It should be noted that the healing abutment can be placed on the dental implant immediately after the implant has been installed and before osseointegration, thereby, for some situations, combining the osseointegration step and gingival healing step into a one-step process.

Prior healing abutments were generally round in profile, but the artificial teeth or prostheses that eventually replaced the healing abutments were not. Thus, the gingival tissue would heal around the healing abutments creating a gingival emergence profile that approximated the size and contour of the healing abutment and not the size and contour of the final prosthesis that was eventually attached to the implant. The resulting discrepancies between the emergence profile of the patient's gingiva and the installed final prosthesis could sometimes require additional visits with the dentist or clinician to finalize the installation process and/or compromise the aesthetic outcome of the installed final prosthesis (e.g., the visual look of the patient's gingival tissue abutting the final prosthesis). Thus, in recent years, standard healing abutments have been replaced with temporary prosthetic abutments.

Further, implant dentistry restorative methods have advanced beyond requiring a fixture-level (e.g., dental implant level) impression as the starting point for developing a final dental prosthesis. In some such cases pre-defined scan bodies (e.g., Encode Healing Abutments available from Biomet 3i, LLC) are assembled to the dental implants during the gingival healing stage. The pre-defined scan bodies include scannable features (e.g., markers) that, when scanned and interpreted, provide information about the location and orientation of the underlying dental implant that is used in developing the final dental prosthesis.

Although such methods using pre-defined scan bodies provide many benefits (e.g., improved aesthetics, reduced complexity, and potentially accelerated treatment times), such methods are reliant on scanning technology. Current predefined scan bodies used in or captured as part of generating a 3D virtual image of all or a portion of the patient's mouth are generally not anatomic, size and shape limited, and are unable to be provisionalized. A need exists for a patient-specific restorative solution that does not require dedicated pre-defined scan bodies as to further reduce the treatment complexity and improve restorative flexibility. There is a need for a patient-specific solution, whether for a predefined scan body configured as a healing collar, temporary tooth or any other type of prosthesis.

### SUMMARY

These and other needs are addressed by the various embodiments and configurations of the present disclosure.

In an embodiment of the present disclosure, a qualification scan assembly can include an attachment surface comprising a bore and a structure positioned in the bore and configured to engage a scan fixture calibration object. The attachment surface can include a first identification code associated with a dental service provider or dental patient and the scan fixture calibration object a second identification code associated with a virtual counterpart of the scan fixture calibration object.

In some embodiments, an attachment member scan assembly can include an attachment surface comprising a bore and a structure positioned in the bore and configured to engage an attachment member and comprising a nonrotational structure. The attachment member can be a patient-specific temporary prosthesis, gingival former, healing cap, healing abutment, (final) abutment or permanent connector for a prosthetic tooth configured to engage an implant in an oral cavity of the patient.

In some embodiments, the structure is a nonrotational structure that may be provided by a dental analog positioned in the bore.

The scan fixture calibration object can be configured as a scan body.

The first and second identification codes are typically different from each other.

The attachment surface can include one or more scannable features to orient one or both of a reference coordinate system and nonrotation structure.

The qualification scan assembly can include a body engaging the attachment surface, the body configured to engage a scanning device that rotates and/or translates the qualification scan assembly during scanning.

In some embodiments, a computational system includes a processor and computer readable medium comprising instructions that, when executed, cause the processor to perform the method of:
receiving fixture scan data comprising an image of a scan fixture engaging a scan fixture calibration object and a dental analog having a nonrotational structure orientation;
generating, from at least a portion of the fixture scan data, a virtual three-dimensional model of the scan fixture and scan fixture calibration object; and
replacing, in the virtual attachment member three-dimensional model, the image of the scan fixture with a virtual counterpart of the scan fixture calibration object to form a modified virtual three-dimensional fixture model comprising one or more of a reference coordinate system position relative to a surface of the scan fixture and an orientation of the nonrotational structure relative to a surface of the scan fixture.

In some embodiments, a computational system includes a processor and computer readable medium comprising instructions that, when executed, cause the processor to perform the method of:
receiving oral scan data of a patient's oral cavity comprising an image of an attachment member attached to a dental implant;
generating, from at least a portion of the oral scan data, a virtual three-dimensional restorative model of the oral cavity;
shape matching a supragingival image of at least a portion of the attachment member with a supragingival virtual counterpart of the at least a portion of the attachment member, each of the supragingival image and virtual counterpart excluding an occlusal surface of the attachment member, and
replacing, in the virtual three-dimensional restorative model, the attachment member with the shape matched virtual counterpart of the attachment member to form a modified virtual three-dimensional restorative model comprising one or more of a seating surface of the attachment member on the dental implant, a reference coordinate system position relative to the seating surface and the orientation of the nonrotational structure relative to the seating surface.

The instructions, when executed, can cause the processor to receive attachment member scan data comprising an image of the scan fixture engaging an attachment member and the dental analog having the nonrotational structure orientation, generate, from the attachment member scan data, a virtual three-dimensional attachment member model of the scan fixture and attachment member, and replace, in the virtual three-dimensional attachment member model, the image of the scan fixture with a virtual counterpart of the scan fixture in the modified virtual three-dimensional fixture model to form a modified virtual three-dimensional attachment member model comprising one or more of a reference coordinate system position relative to a surface of the attachment member and the orientation of the nonrotational structure relative to a surface of the attachment member.

The instructions, when executed, can cause the processor to receive oral scan data of a patient's oral cavity comprising an image of the attachment member attached to a dental implant, generate, from the oral scan data, a virtual three-dimensional restorative model of the oral cavity, and replace, in the virtual three-dimensional restorative model, the image of the attachment member with a virtual counterpart of the attachment member in the modified virtual three-dimensional attachment member model to form a modified virtual three-dimensional restorative model comprising one or more of a seating surface of the attachment member on the dental implant, a reference coordinate system position relative to the seating surface and the orientation of the nonrotational structure relative to the seating surface.

The instructions, when executed, can cause the processor to remove the image of the attachment member from the virtual three-dimensional restorative model to provide the modified virtual three-dimensional restorative model.

The instructions, when executed, can cause the processor to determine a degree of mismatch between an outer contour of the attachment member in the virtual three-dimensional restorative model and the modified virtual three-dimensional attachment member model and, when the degree of mismatch is within a predetermined range, determining that the virtual three-dimensional restorative model is invalid and repeating the receive and generate operations with a second set of oral scan data.

In some embodiments, a computer readable medium includes plural libraries comprising a plurality of records. Each record includes an identifier of a scan fixture and virtual 3D models of the scan fixture and attachment member associated with the identifier. Each library is associated with a unique identifier of a restoration service provider, patient and/or tooth.

Each scan fixture identifier can be associated with a specific type of nonrotational structure. The virtual 3D models of the scan fixture and attachment member can comprise a representation of the nonrotational structure.

The present disclosure can provide a number of advantages depending on the particular configuration.

The present disclosure can develop and fabricate provisional and permanent patient-specific prostheses without needing pre-defined scan bodies. Thus, the methods of the present disclosure can reduce treatment complexity and enhance restorative flexibility and thereby improve the dental restoration process. A patient-specific temporary prosthesis (PSTP) or attachment member can be fabricated and scanned to generate PSTP scan data and/or a virtual three-dimensional model of the PSTP that captures shape matchable contours and details of the PSTP. The PSTP is attached to the implant in the patient's mouth and the gingival tissue is permitted to heal therearound. When a clinician determines that the gingival tissue has healed around the PSTP in a desired manner (e.g., aesthetically pleasing manner), a permanent patient-specific prosthesis is created as a replica of the PSTP using the scan data and/or the virtual three-dimensional model of the PSTP. By scanning the PSTP and generating scan data and/or the virtual three-dimensional model of the PSTP, neither are predefined scan bodies necessary to develop and fabricate the permanent patient-specific prosthesis nor are pre-defined scan bodies necessary to determine the location of the implant with respect to the adjacent and/or opposing dentition.

While there are hundreds of unique encode healing abutments available today, their application is limited because they are not offered in unlimited shapes and their function during healing is to simply shape the tissue (i.e., it does not serve as a tooth prosthesis). With the present disclosure and knowing the utilization of dental implants, it is clear that clinicians, as part of the workflow described herein, could create and scan 500,000 to 10,000,000 or more patient-specific attachment members per year configured as healing collars, temporary teeth or any other type of prostheses. Knowing that each of these attachment members, scan fixtures and the patient datasets would need to be stored in a database, scan fixture and data management is very important, as is the accuracy of the scan fixtures and attachment members.

Scan fixtures, scan fixture calibration objects, and attachment members of the present disclosure can be not only patient-specific but also easy to design and fabricate. Attachment members can be efficiently and flexibly configured as contoured to have the appearance of a tooth that can be used in the anterior of a patient's mouth. The scan fixture, scan fixture calibration object, and attachment member can be 3D printed on site by the restoration service provider. The attachment member can enable a more accurate capture of the emergence profile as it enables profile capture from the tissue without tissue disruption (thereby avoiding under contouring or under support of the tissue) versus the tooth as in conventional healing abutments. The attachment member can be encoded with information markers to translate data about the emergence profile and underlying 3D coordinate system used in the virtual 3D model of the selected portion of the patient's mouth. However, unlike a normal encoded healing abutment, there is generally no need for information markers, such as “dimples and divots”, on the top surface to translate data about the emergence profile and underlying 3D coordinate system. This can avoid the use of complicated libraries or library management, particularly since decryption of the scan files is performed in the cloud by the modeling system.

The qualification and restorative scan assemblies can enable the creation of an accurate virtual 3D model of a selected portion of a patient's mouth undergoing restoration that fixes the attachment member in five or six degrees of freedom depending on the presence or absence of constraints in the connection interface between the implant and attachment member.

These and other advantages will be apparent from this disclosure.

The phrases “at least one”, “one or more”, “or”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C”, “A, B, and/or C”, and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The term “computer-readable medium” as used herein refers to any computer-readable storage and/or transmission medium that participate in providing instructions to a processor for execution. Such a computer-readable medium can be tangible, non-transitory, and non-transient and take many forms, including but not limited to, non-volatile media, volatile media, and transmission media and includes without limitation random access memory (“RAM”), read only memory (“ROM”), and the like. Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

A “computer readable storage medium” may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may be any computer readable medium that is not a computer readable storage medium and that can communicate, propagate, or transport a program for use by or in connection with an instruction execution system, apparatus, or device. A computer readable signal medium may convey a propagated data signal with computer readable program code embodied therein, for example, in baseband or as part of a carrier wave. Such a propagated signal may take any of a variety of forms, including, but not limited to, electro-magnetic, optical, or any suitable combination thereof. Program code embodied on a computer readable signal medium may be transmitted using any appropriate medium, including but not limited to wireless, wireline, optical fiber cable, RF, etc., or any suitable combination of the foregoing.

The terms “determine”, “calculate” and “compute,” and variations thereof, as used herein, are used interchangeably and include any type of methodology, process, mathematical operation or technique.

The term “means” as used herein shall be given its broadest possible interpretation in accordance with 35 U.S.C., Section(s) 112(f) and/or 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “module” as used herein refers to any known or later developed hardware, software, firmware, artificial intelligence, fuzzy logic, or combination of hardware and software that is capable of performing the functionality associated with that element.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein. By way of example, the phrase from about 2 to about 4 includes the whole number and/or integer ranges from about 2 to about 3, from about 3 to about 4 and each possible range based on real (e.g., irrational and/or rational) numbers, such as from about 2.1 to about 4.9, from about 2.1 to about 3.4, and so on.

The preceding is a simplified summary of the disclosure to provide an understanding of some aspects of the disclosure. This summary is neither an extensive nor exhaustive overview of the disclosure and its various embodiments. It is intended neither to identify key or critical elements of the disclosure nor to delineate the scope of the disclosure but to present selected concepts of the disclosure in a simplified form as an introduction to the more detailed description presented below. As will be appreciated, other embodiments of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present invention(s). These drawings, together with the description, explain the principles of the invention(s). The drawings simply illustrate preferred and alternative examples of how the invention(s) can be made and used and are not to be construed as limiting the invention(s) to only the illustrated and described examples. Further features and advantages will become apparent from the following, more detailed, description of the various embodiments of the invention(s), as illustrated by the drawings referenced below.

FIG. 1 is a block diagram of a networked dental restoration system according to an embodiment of the present disclosure;

FIG. 2A is an isometric view of a scan fixture according to an embodiment of the present disclosure;

FIG. 2B is a top view of the scan fixture of FIG. 2A;

FIG. 2C is a side view of a scan fixture showing an analog before positioning in a central bore of the scan fixture;

FIG. 2D is a side view of the scan fixture showing the analog after positioning in the central bore of the scan fixture;

FIG. 3A is a side view of a scan fixture mounting assembly according to an embodiment of the present disclosure;

FIG. 3B is a top view of the scan fixture mounting assembly of FIG. 3A;

FIG. 3C is a top view of the scan fixture mounting assembly of FIG. 3A while engaging a scan fixture;

FIG. 4 is an isometric view a scan fixture with a scan fixture calibration object engaging the analog positioned in the central bore;

FIG. 5 is a block diagram of a dental service provider computational device according to an embodiment of the present disclosure;

FIG. 6 is a block diagram of a dental service provider computational device according to an embodiment of the present disclosure;

FIG. 7 is a side view of an intraoral scanner scanning a scan fixture and scan fixture calibration object while mounted in the scan fixture mounting assembly of FIGS. 3A and 3B according to an embodiment of the present disclosure;

FIG. 8 is a screen shot of a fixture scan data according to an embodiment of the present disclosure;

FIG. 9 is a screen shot of a scan fixture 3D model after removal of the scan fixture calibration object;

FIG. 10 is a process workflow according to an embodiment of the present disclosure;

FIG. 11 is a screen shot of a scan fixture 3D model before removal of the scan fixture calibration object;

FIG. 12 is an isometric view of an attachment member mounted on an analog of a scan fixture according to an embodiment;

FIG. 13 is a screen shot of attachment member scan data according to an embodiment of the present disclosure;

FIG. 14 is a screen shot of an attachment member 3D model according to an embodiment of the present disclosure;

FIG. 15 is a screen shot of a restorative 3D model before removal of the attachment member according to an embodiment of the present disclosure;

FIG. 16 is a screen shot of a restorative 3D model before removal of the attachment member according to an embodiment of the present disclosure;

FIG. 17 is a screen shot of a is a screen shot of a restorative 3D model after removal of the attachment member according to an embodiment of the present disclosure;

FIG. 18 is a screen shot of a is a screen shot of a restorative 3D model after removal of the attachment member according to an embodiment of the present disclosure;

FIG. 19 is an isometric view of an abutment and implant according to an embodiment of the present disclosure;

FIG. 20 is a screen shot of a restorative 3D model after removal of the attachment member according to an embodiment of the present disclosure;

FIG. 21 is a screen shot of a restorative 3D model after removal of the attachment member according to an embodiment of the present disclosure;

FIG. 22 are views of an attachment member attached to analog in a first case study according to an embodiment of the present disclosure;

FIG. 23 is a side view of an intraoral scanner scanning a scan fixture and attachment member while mounted in the scan fixture mounting assembly of FIGS. 3A and 3B in the first case study according to an embodiment of the present disclosure;

FIG. 24 is a screen shot of oral scan data in the first case study according to an embodiment of the present disclosure;

FIG. 25 is a screen shot of a restorative 3D model before removal of the attachment member in the first case study according to an embodiment of the present disclosure;

FIG. 26 is a screen shot of the restorative 3D model of FIG. 25 after removal of the attachment member in the first case study according to an embodiment of the present disclosure;

FIG. 27 is a screen shot of the restorative 3D model of FIG. 26 in the first case study according to an embodiment of the present disclosure;

FIG. 28 is a perspective view of an attachment member being engaged with an analog in a second case study according to an embodiment of the present disclosure;

FIG. 29 is a side view of an intraoral scanner scanning a scan fixture and attachment member while mounted in the scan fixture mounting assembly of FIGS. 3A and 3B in the second case study according to an embodiment of the present disclosure;

FIG. 30 is a screen shot of attachment member scan data in the second case study according to an embodiment of the present disclosure;

FIG. 31 is a screen shot of a restorative 3D model before removal of the attachment member in the second case study according to an embodiment of the present disclosure;

FIG. 32 is a screen shot of the restorative 3D model of FIG. 31 after replacement of the attachment member with the scan fixture calibration object in the second case study according to an embodiment of the present disclosure;

FIG. 33 depicts multiple attachment members before engagement with corresponding analogs in a physical model of a patient's oral cavity in a third case study according to an embodiment of the present disclosure;

FIG. 34 depicts multiple attachment members after engagement with corresponding analogs in the physical model of the patient's oral cavity in the third case study according to an embodiment of the present disclosure;

FIG. 35 is a screen shot of the restorative 3D model before removal of the multiple attachment members in the third case study according to an embodiment of the present disclosure;

FIG. 36 is a plan view of the emergent profile of a portion of the physical model of the third case study;

FIG. 37 is a screen shot of the restorative 3D model with the abutments in position in the third case study according to an embodiment of the present disclosure; and

FIG. 38 is an isometric view of a rapid prototype model containing multiple abutments corresponding to the tooth positions of the multiple attachment members in the third case study.

### DETAILED DESCRIPTION

The present disclosure describes a scan assembly comprising a uniquely encoded scan fixture, encoded scan fixture calibration object, and (unencoded) patient-specific, provisionalized attachment member (e.g., temporary restoration or patient-specific temporary prosthesis (PSTP) or patient-specific abutment) that can be used in conjunction with other images to generate virtual 3D images of the patient's mouth to enable design of a dental restoration. In a registration, calibration, or qualification mode, the scan fixture calibration object is engaged with the scan fixture (in the absence of the attachment member or PSTP) and scanned outside the patient's mouth to provide precise and accurate scanning information about the scan fixture, including conveying the position of a virtual 3D coordinate system and/or position and/or orientation of the underlying connection interface (e.g., nonrotational structure). In an attachment member scanning mode, the attachment member is engaged with the scan fixture (in the absence of the scan fixture calibration object) and scanned outside the patient's mouth to enable the virtual 3D coordinate system and connection interface orientation to be imparted to the attachment member for later use in generating the virtual 3D images of the patient's mouth. The use of the registration or qualification mode can provide increased accuracy in positioning a virtual counterpart of the attachment member in the virtual 3D coordinate system and the unique encoding of the scan fixture can link various scanned images to a restoration service provider, specific tooth position undergoing restoration, and/or patient. The attachment member can be configured as any desired dental component, including as a healing abutment or temporary tooth.

The present disclosure describes a digital processing workflow that can receive scan images of the scan fixture and scan fixture calibration object (e.g., the qualification scan assembly) and of the scan fixture and attachment member (e.g., the restorative scan assembly) and, based on the scan images, determine a virtual 3D coordinate system for the attachment member engaged with the scan fixture and other information including, without limitation, the location of a seating surface or connection interface between the attachment member and scan fixture and position and orientation of a nonrotational structure in the connection interface relative to the coordinate system. The virtual 3D coordinate system can be imparted to virtual 3D models created through scanned images of a patient's mouth comprising the attachment member (in the absence of the scan fixture). The shape or contours of the attachment member can also be captured in the scanned images of the restorative scan assembly and shape matched with the shape or contours of the attachment member in intraoral scanned images of the attachment member in the patient's mouth to select a virtual counterpart of the attachment member from a database library. The selected virtual counterpart can replace the scanned attachment member in the intraoral scanned images during virtual 3D model generation.

A unique code or other identifier can be located on the scan fixture to enable the scan fixture to be mapped to a particular restoration service provider (e.g., case identifier), tooth undergoing restoration (e.g., tooth identifier), and/or patient (e.g., patient identifier) and an associated database library comprising scans of the qualification and restorative scan assemblies and optionally a virtual counterpart of the attachment member. Typically, the latter two items, namely the tooth undergoing restoration and/or patient, would be entered by the service provider into the cloud when the service provider uploads the scan data, which are then associated with a scan fixture registered to the corresponding service provider. When scanned images are received from the restoration service provider, they can be automatically paired with the database library containing previously received scanning information associated with the qualification and restorative scan assemblies. While a unique code can be employed, it is not a requirement. It is simply a way to automate and/or make the workflow more efficient. In reality, the scan fixtures could all be the same and the scanned data can be stored manually (basically, this would be a type of brute force approach).

A restorative scan assembly can include the scan fixture and an attachment member, whether in the form of a healing abutment or temporary tooth.

The present disclosure further describes an optical scanning device and method for scanning the scan fixture, attachment member, or qualification and restorative scan assemblies. Encode healing abutments today are stock and go from the package to the patient's mouth with little thought. While the scan assembly and attachment member of the present disclosure is a significant step forward, it does require the clinician or lab to perform an extra step of scanning each and every scan assembly prior to placement or after a component has been modified. To make this process both simple, inexpensive and robust, an intra-oral scanner can be provided or enable the scan of the scan fixture and scan assembly to be done with a smart phone, tablet computer, or other personal communication device. Rather than having the customer scan these conventionally, which means rotating the scanner around and about (in the present case) the scan fixture or scan assembly, a simple stepper motor with a socket for the scan fixture or scan assembly can be provided. Additionally, the socket can be configured to the stepper motor to not only rotate but also move up and/or down to aid in the capturing of the scan data. The clinician can hold the intra-oral scanner or camera next to the scan fixture or scan assembly/motor set up and push a button which would rotate the motor one or more revolutions. Alternatively, an additional fixture that holds both the motor and camera or scanner is provided so that the distance and orientation of the optical device relative to the scan fixture can be controlled.

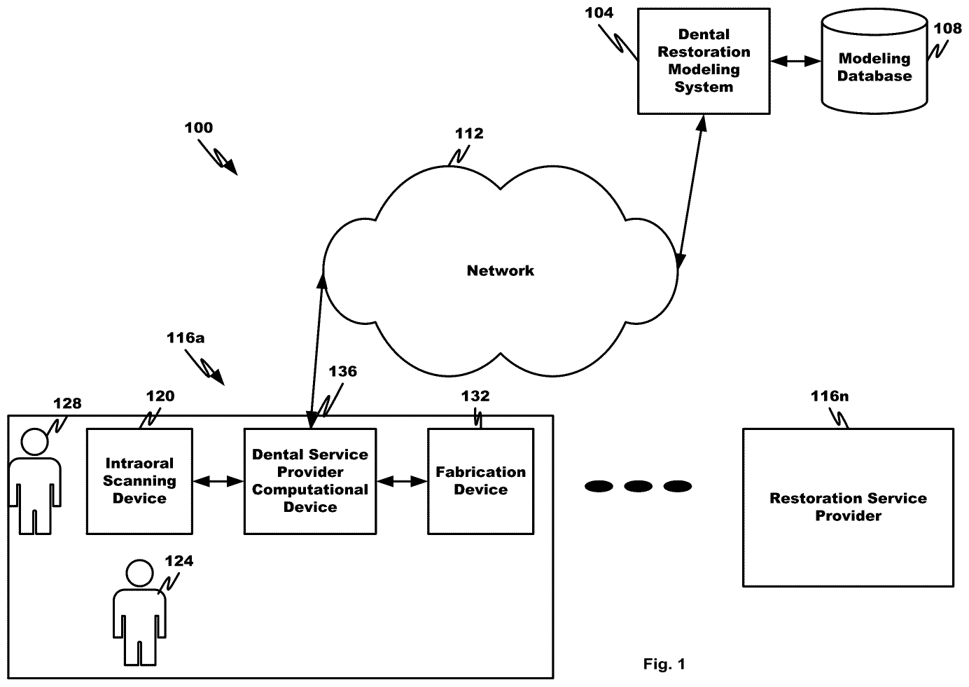

FIG. 1 depicts a networked dental restoration system 100 according to an embodiment of the present disclosure. The system 100 comprises a dental restoration modeling system 104 and associated modeling database 108 interconnected via a network 112 with a plurality of restoration service providers 116a-n (such as a dental laboratory, dental surgical facility, or dental office). Each dental service provider 116 includes an intraoral scanning device 120 operated by a technician 124 for scanning an oral cavity of a patient 128 and a fabrication device 132 to fabricate dental components, both of which are in electrical communication with a dental service provider computational device 136.

The network 112 may correspond to a distributed set of devices that interconnect and facilitate machine-to-machine communications between the components of the system 100. The network 112 may include any type of known communication medium or collection of communication media and may use any type of protocols to transport messages between devices. The Internet is an example of the network 112 that constitutes an IP network consisting of many computers, computing networks, and other communication devices located all over the world, which are connected through many telephone systems and other means. Other examples of the network 112 include, without limitation, a standard Plain Old Telephone System (POTS), an Integrated Services Digital Network (ISDN), the Public Switched Telephone Network (PSTN), a cellular network, and any other type of packet-switched or circuit-switched network known in the art.

The intraoral scanning device 120 may be any device or set of devices for generating a scan of a portion of the patient's oral cavity. The intraoral scanning device can be any handheld device that captures digital images of the oral cavity by projecting light (e.g., structured light or a laser) onto the teeth and surrounding tissues. As the light contacts the teeth and surrounding tissues, it distorts and high-resolution cameras in the wand capture these distortions, creating a detailed 3D model in real-time. The scanning device takes thousands of images per second from various angles and analyzes and stitches together the images to create a detailed 3D representation of the oral cavity.

The fabrication device 132 receives commands from the computational device 136 and fabricates dental components from the generated 3D models. The device 132 can be an additive or subtractive fabrication device, such as a 3D printer, or a milling device. Exemplary 3D printers include fused deposition modeling printers, resin 3D printers, selective laser sintering printers, direct metal laser sintering printers, electron beam melting printers, binder jetting printers, multi jet fusion printers, material jetting or polyjet printers, directed energy deposition printers, and laminated object manufacturing or selective deposition lamination printers.

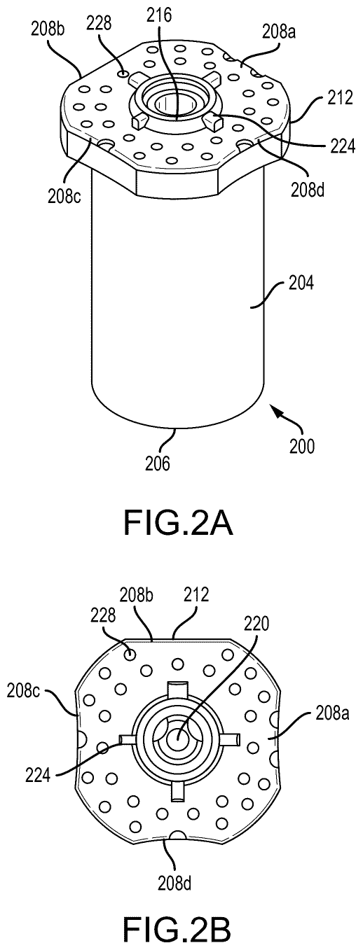

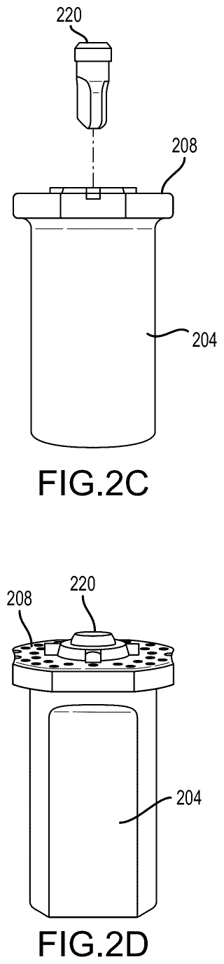

Referring to FIGS. 2A-D and 4, a qualification scan assembly includes a scan fixture 200 and a scan fixture calibration object 400. The scan fixture 200 can further include a body 204, a base 206, and an attachment interface 208 having an outer peripheral edge 212. The body and outer edge 212 can have any desired shape. The attachment interface 208 further includes a central bore 216 to receive an analog 220 to matingly engage a projection 404 (e.g., a threaded screw) of a scan fixture calibration object 400. The analog 220 is typically associated with a particular implant and nonrotational structure that may matingly engage an attachment interface of the scan fixture calibration object or attachment member. The analog 220 can include a nonrotational or anti-rotational structure to inhibit rotation of the attachment member relative to the scan fixture. The nonrotational structure of the projection 404 is configured to mate in a slideable engagement with a corresponding nonrotational structure of the analog to prevent relative rotation of the scan fixture calibration object.

As will be appreciated, there are many types of nonrotational or anti-rotational features or structures depending on the implant manufacturer and application (e.g., hex-shaped (associated with Zim Vie™, Nobel Biocare™, and BioHorizons™), square-shaped (associated with Staumann™), star-shaped (e.g., Keystone Dental), and the like. As will be appreciated, a separate set of virtual 3D scan fixture models will need to linked to a corresponding scan fixture identifier for each type of feature or structure.

While the present disclosure is discussed in connection with such features or structures, it is to be understood that the teachings of this disclosure may apply to dental systems that do not employ such features or structures, such as non-engaging abutments (that do not have an anti-rotational feature and can rotate on the implant).

The scan fixture calibration object 400 can have any shape and size depending on the requirements of the qualification process. Typically, the scan fixture calibration object 400 has one or more irregular outer surfaces or contours to enable an accurate determination of the position and orientation of any nonrotational feature at the connection interface and of the datum plane of the virtual 3D coordinate system relative to the scan fixture. Exemplary scan fixture calibration objects include Zim Vie's GenTek™ scan bodies, among others. Typically, the scan fixture calibration object 400 will have a uniform size and shape from patient to patient and be differently sized and shaped than the attachment member.

Both the upper surfaces 224 and 408 of the scan fixture and scan fixture calibration object can include a unique code 228 and 412, respectively. The unique code on the upper surface 224 of the scan fixture associates the scan fixture with a particular restoration service provider (e.g., case identifier), tooth undergoing restoration (e.g., tooth identifier), and/or patient (e.g., patient identifier) or case (e.g., case identifier) and an associated database library comprising scans of the qualification and restorative scan assemblies and optionally a virtual counterpart of the attachment member. In contrast, the unique code on the upper surface 408 of the scan fixture calibration object is associated with a virtual counterpart of the scan fixture calibration object in a library comprising multiple different sizes and/or types of scan fixture calibration objects.

The upper surfaces of the scan fixture calibration object and scan fixture can include additional information. The code on the upper surface of the scan fixture calibration object can include a binary-coded marker (e.g., the presence or absence of a marker indicates the calibration object's height, diameter, dimension of the attached analog seating surface, and/or orientation of the analog's non-rotational structure relative to a reference coordinate system). Likewise, the upper surface of the scan fixture can include one or more markers 224 (shown as outwardly radiating ridges surrounding the central bore) and/or differently configured peripheral edge segments 208a-b that provide reference features indicating a position of the datum plane of the virtual 3D coordinate system relative to the scan fixture and/or an orientation of the non-rotational structure of the analog relative to the datum plane or reference coordinate system. As can be seen from FIG. 2B, the edge segment 208a has two notches, edge segment 208b is without notches, and edge segment 208c and d each has one notch, such that opposing pairs of edges form differently configured edge pairs.

The codes or identifiers on the upper surfaces 224 and 404 of the scan fixture and scan fixture calibration object, respectively, can be the same or different from each other. The code or identifier can be any unique scannable set of features, including negative surface features such as dimples, notches, etched lines, grooves, other recessed features, and the like, positive surface features such as pimples, bumps, ridges, raised lines, other raised features, and the like, etched or printed QR or other bar codes, color, shape or outer contour or peripheral surface, and any other feature that is recognizable in the scan data (e.g., that can be recognized by shape matching against one or more virtual scan fixture or scan fixture calibration object counterparts, as appropriate, in a virtual library of virtual counterparts).

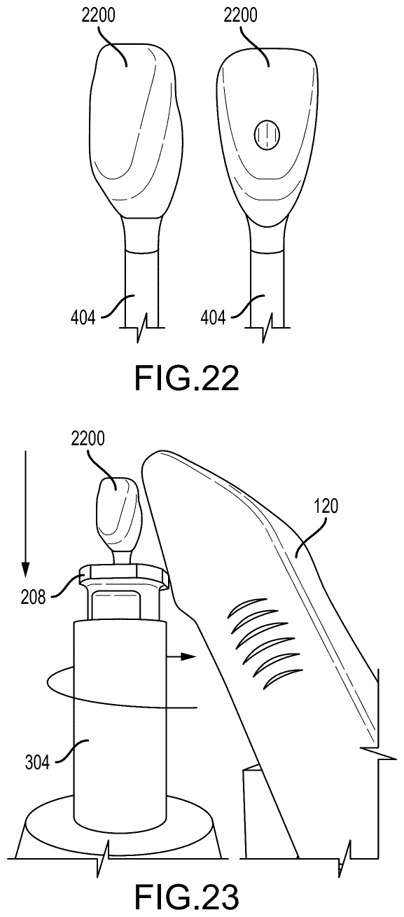

Referring to FIGS. 12 and 22, the attachment member 1200, 2200 can have any shape and size depending on the application. In some applications, the attachment member 1200, 2200 is configured as a conventional healing abutment. Exemplary healing abutments include Zim Vie's Encode™ emergence healing abutments, Eztetic™ healing abutments, BellaTek encode healing abutments, TSX™ or TSV™ healing abutments, and the like. In some applications, the attachment member 1200, 2200 is configured as a tooth customized to appear as a natural tooth in the patient's mouth that is particularly attractive for anterior restorations. Unlike the prior application, the customized attachment member 1200, 2200 can vary in shape and/or size from patient to patient. FIG. 12 shows the attachment member attached to a titanium base 1204, and the titanium base is assembled or attached to the scan fixture.

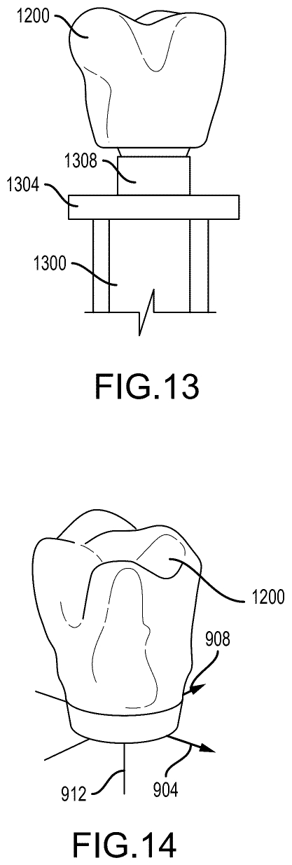

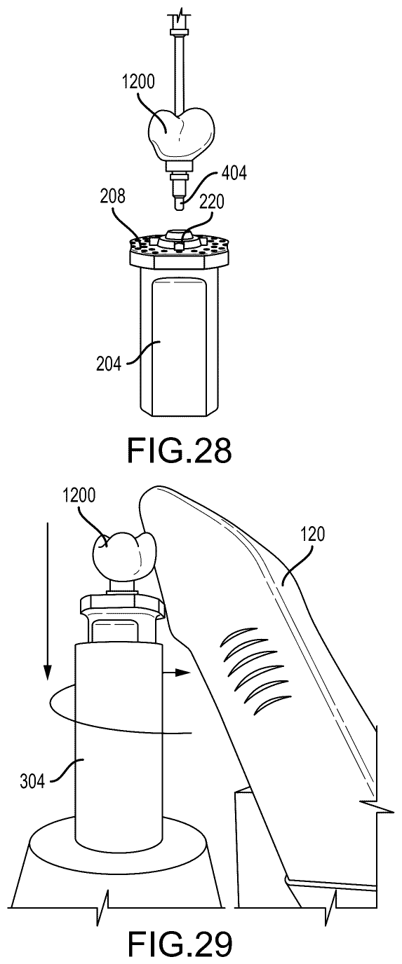

The attachment member 1200, 2200 can be scanned either after it is attached (e.g., cemented, screwed into, or otherwise interconnected) to the scan fixture, such as a titanium base with a temporary crown or healing collar, or processed from a monolithic abutment (either as is or modified by material addition or removal). Typically, the attachment member 1200, 2200 is assembled to the scan fixture 200 to form the restorative scan assembly as shown in FIGS. 28 and 29. Referring to FIGS. 28-29, the attachment member 1200 is engaged with the analog 220 in the scan fixture 200, and the body 204 is placed in the scan fixture receiver 304. The stepper motor 312 is activated and the scanning device scans the attachment member during rotation and translation as shown by the arrows. An example of the attachment member scan data is shown in FIG. 13, which shows a captured image of the scan fixture 1300, connection interface 1304, titanium base 1308, and attachment member 1200.

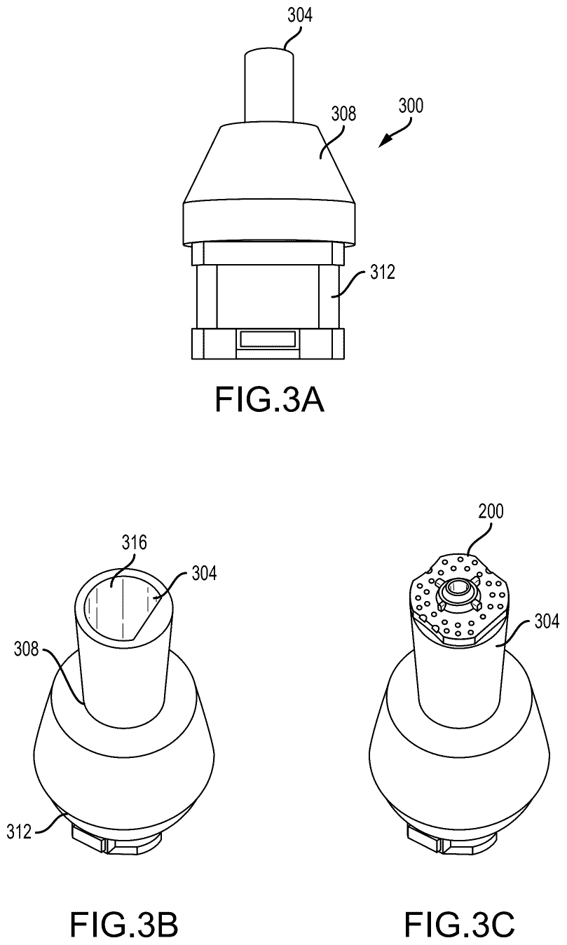

With reference to FIGS. 3A-3C, the present disclosure further describes an optical scanning device and method for scanning the scan fixture, attachment member, or qualification and restorative scan assemblies. Referring to FIGS. 3A-3C, a scan fixture mounting assembly 300 comprises a scan fixture receiver 304 engaged with a rotatable housing 308 engaged with a stepper motor 312. As can be seen from FIGS. 3B and 3C, the scan fixture receiver 304 comprises a bore 316 having an inner shape to match an outer shape of the body 204 of the scan fixture 200 such that the body is matingly received by the bore 316 as depicted in FIG. 3C. As will be appreciated, the scan fixture receiver 304 can alternatively comprise an outer shape that is received in a similarly shaped bore in the body 204 of the scan fixture 200

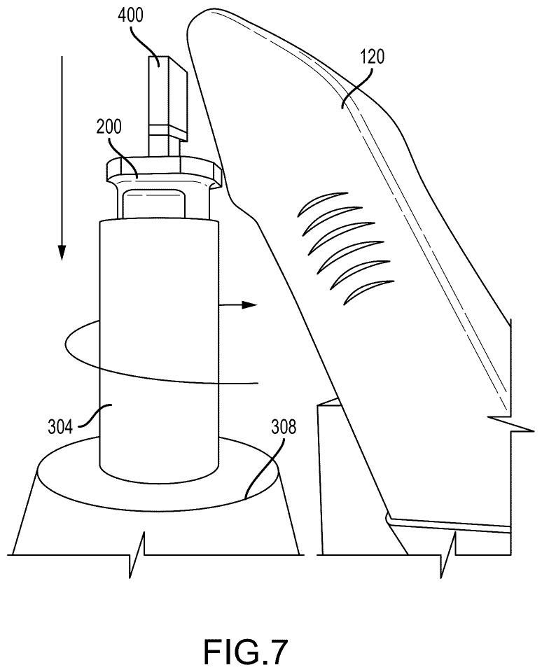

FIG. 7 depicts the scanning method used with the optical scanning device. With reference to FIG. 7, an optical or intraoral scanning device 120 is positioned facing the scan fixture calibration object 400 while the stepper motor 312 rotates the scan fixture receiver and moves the scan fixture upwardly and downwardly as shown by the arrows to aid in the capturing of the scan data while the scanning device 120 is in the scanning mode. The upward and downward movement can be effected by a rack and pinion gear system (not shown) or other suitable type of gear. As will be appreciated an additional fixture holding both the stepper motor and scanning device may be employed to enable control of the distance and orientation of the scanning device relative to the scan fixture.

With reference to FIG. 9 by scanning the scan fixture alone and/or with the scan fixture calibration object and/or with the attachment member attached, the virtual 3D coordinate system 900 comprising Z, Y, and Z axes 904, 908, and 912 for the attachment interface between the attachment member and scan fixture can be determined before the attachment member 1200, 2200 is connected to an implant (comprising the same connection interface as the scan fixture) in the patient's mouth. As will be appreciated, the connection interface does not need to be identical but should have the same constraints as the clinician intends to transfer to the patient. Both the male and female sides of the connection interface has different connections for the same interface.

FIG. 9 provides more details on the connection interface 208. The code 228 is shown to include a plurality of dimples 916 uniquely arranged to provide a unique identification code. Additionally, the notches 920 on the various peripheral edge segments are shown. The ridges radiating from the bore are also shown.

Referring to FIG. 14, the reference coordinate system can be translated by the attachment member 3D model from the virtual connection interface 208 to the virtual attachment member. In the screenshot of FIG. 14, the virtual scan fixture has been removed by the 3D model to display the image of the virtual attachment member only. The virtual attachment member can provide the same information and perform the same function as a healing abutment except that is fully patient specific.

While the virtual counterpart of the scan fixture 200 can be removed from the restorative scan assembly to yield the virtual counterpart of the attachment member 1200, other techniques can be employed. Since the locations of the coordinate system and corresponding datum plan are known, the workflow can isolate the virtual counterpart of the attachment member 1200 simply by showing the image information above the datum plane (e.g., the virtual attachment member 1200) or alternatively hiding the image information below the datum plane (e.g., the scan fixture 200).

After the attachment member 1200, 2200 is positioned in the patient's mouth and the gingiva has healed the clinician 124 can scan the patient 128 just as if he or she has a conventional attachment member in the same position. The digital workflow can overlay a custom encode database library file associated with a specific restoration service provider and/or patient and containing an identifier of a scan fixture, scans of the restorative scan assembly comprising the scan fixture, and optionally a virtual counterpart of the scan fixture, restorative scan assembly containing the scan fixture, and/or attachment member connected to the restorative scan assembly (and optionally generated by removing a virtual counterpart of the scan fixture from a virtual counterpart of the restorative scan assembly) associated with previously uploaded scans with the subsequent patient scan data.

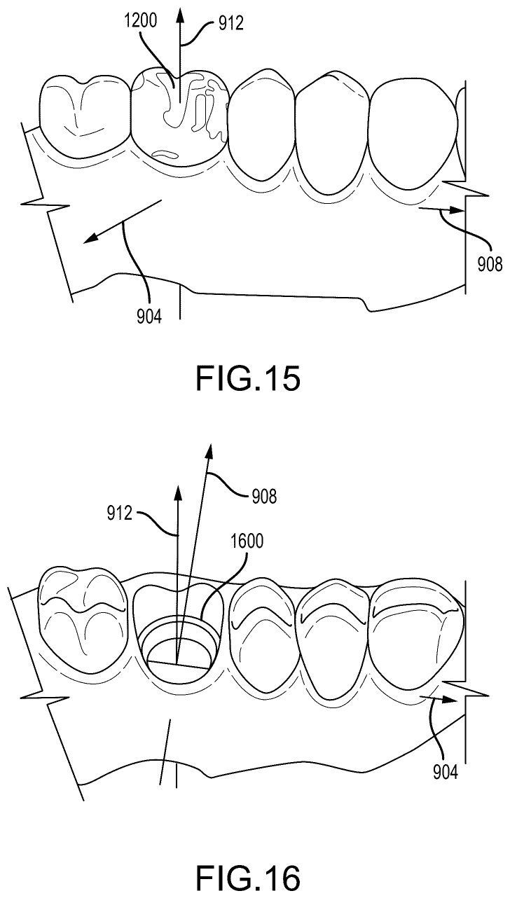

With reference to FIGS. 15 and 16 (which show displays output by the restorative 3D model) because the coordinate system 900 is known and is the same coordinate system for the connection interface 208, the attached scan fixture calibration object, the attached attachment member, and the implant, the coordinate system can be generated in the virtual 3D image of the patient's mouth used for the restoration, and the attachment member captured in the oral scan data can be deleted or otherwise removed by the digital workflow to yield a virtual 3D model comprising only a virtual counterpart 1500 of the attachment member engaged with a virtual counterpart of the implant or of the seating surface 1600 of the implant with the virtual counterpart 1500 removed. In other words, the restorative scan assembly produces a similar virtual 3D model to conventional healing abutments but can be fully patient customizable, such as being configured as a temporary tooth for anterior installment.

In other words, the virtual attachment member is removed from the virtual 3D model in the same manner as is done with a conventional healing abutment. Once removed, the modified virtual 3D model indicates the position of the connection interface and relative position of the nonrotational structure of the underlying implant and provides the appearance of the sub-gingival tissue (or the emergence profile).

Scan assembly and data management can be done in a variety of ways. It is desirable that the management be robust and reliable so as to (a) “encode” the patient-specific “fully customizable” attachment member and (b) manage the “single” database library files. For example, the attachment member can be 3D printed and permanently bonded to each known analog connection (or nonrotational structure) in each of the printed scan fixtures. Alternatively, a high precision scan fixture can be made for each known implant connection.

In some applications, based on the assumption that the 3D printed scan fixtures 200 can have a high risk of being inaccurate, especially when compared to a precision machined scan fixture 200, the various scan fixtures are first “set up” and calibrated by a qualification scan assembly. To do this, a very high precision scan fixture calibration object 400 for each known implant or analog connection is provided, which would then be assembled to the analogs 220 (or connection interfaces) within the scan fixtures 200 to provide plural qualification scan assemblies. The clinician would scan each of these qualification scan assemblies and upload the scan images for digital processing. The processor during digital processing would identify the virtual 3D coordinate system for each selected qualification scan assembly, the location of the connection interface on the qualification scan assembly, and orientation of the nonrotational structure relative to the coordinate system 900 and, when identified, the qualification scan assembly and associated scan fixture would become “qualified”.

Every scan fixture 200 would be and should be unique. Using the 3D virtual model of the qualified scan fixture (or scan fixture 3D model), printing instructions for the scan fixture 200 would be generated to enable each and every scan fixture 200 to be 3D printed. The scan fixtures are made distinct using a suitable unique identifier, such as the encodings discussed above. A clinician or dental service provider 116 could print multiples of a single file, but only the first file which is scanned in and submitted to the digital processing system will be “qualified”. It is no different than someone trying to use a unique code more than once. After the first file is uploaded and processed, the clinician or dental service provider 116 has a unique fixture identifier, and it is known who the clinician or dental service provider 116 is that has the uploaded scan fixture image and what connection analog has been glued into the scan fixture 200. As will be appreciated, the scan fixtures 200 could alternatively be prefabricated and calibrated known fixtures.

In some embodiments, the unique identifier or other recognizable feature of the scan fixture (e.g., the ridges 224 or notches on the peripheral edge segments 208a-d) acts as an information marker to indicate a relative position of the nonrotational structure of the connection interface 208 relative to the 3D coordinate system 900. As a counterpart of the connection interface of the implant (e.g., the analog 220) is located on the scan fixture 200, the scan of the scan fixture 200 without the attachment member can enable the digital workflow to determine an orientation of the nonrotational structure (which is exposed on the seating surface of the scan fixture) relative to the recognizable feature. When a further scan is performed of the scan assembly, the recognizable feature can be used to determine the position and orientation of the nonrotational structure of the attachment member. In this manner, when a virtual counterpart of the attachment member is selected from the restoration service provider's library by shape matching and positioned in the 3D virtual model by shape matching of the virtual attachment member counterpart against the scanned image of the attachment member attached to the implant in the patient's mouth (shown in the oral scan data), the relative position and orientation of the nonrotational structure can also be determined using the recognizable feature on the image of the scan assembly stored in the library. As will be appreciated, it is possible not to have a nonrotational structure in the attachment members.

The “scan calibration object” 400 or scan body that would be used for qualification should be of a size required to produce the most precise scan. Unlike conventional healing abutments, it would not need to fit into the mouth or between the teeth of a patient. In most applications, the scan calibration object 400 would not fit between the patient's teeth or otherwise into the patient's mouth. It is much easier to provide high-precision scan fixtures for each implant connection (or nonrotational structure) that can be reused vs. high-precision scan fixtures 200 that will wear out over time. It is easy and inexpensive to 3D print scan fixtures on a fabrication device 132 to replace worn out or broken fixtures. The scan fixture 200 would be requalified periodically.

Once a clinician or service provider 116 has qualified scan fixtures 200 for the various implant connections using scan fixture calibration objects 400, he or she can commence scanning in the patient-specific attachment members (e.g., healing abutments and temporary restorations). As he or she scans, the scan images can be uploaded into a converter tool (or this logic can be built into the digital processing software), which would then “encode” the file and save it in a database library associated with the clinician's or restoration service provider's unique identifier for later reference after it has been placed in the patient's mouth, healed and scanned again. In other words, each clinician or restoration service provider would have a uniquely identified database library containing, optionally in patient indexed files, scans of the scan fixtures 200 and attachment members 1200 alone, the previously “qualified” qualification scan assemblies, and the restorative scan assemblies associated with a restoration being performed on the corresponding patient.

As a further point for ease of use, once the clinician or service provide pushes the “go button” on the optical scanning device, the scanning can be automated, and collection of data and submission of the file can be automatically uploaded to the digital processing software. The restoration software manages the conversion and storage of the data until such time the clinician scans the healed patient. Reception of the additional data and the shape matching and decryption can be managed in the cloud for the clinician to make this as seamless a process as possible.

The attachment member can be modified during the healing phase and rescanned. If for some reason there is degradation of or change in the scan geometry during the healing phase, the clinician or service provider 116 scan rescan the scan fixture as to re-encode it to ensure an accurate library file for shape matching.

Referring to FIG. 5, an embodiment of the dental service provider computational device 136 will be described. The dental service provider computational device 136 is shown to include a processor 504, memory 500, a user interface 508, and a network interface 512. In some embodiments, the processor 504 may correspond to one or many microprocessors, CPUs, microcontrollers, Integrated Circuit (IC) chips, or the like. For instance, the processor 504 may be provided as silicon, as a Field Programmable Gate Array (FPGA), an Application-Specific Integrated Circuit (ASIC), any other type of Integrated Circuit (IC) chip, a collection of IC chips, or the like. As a more specific example, the processor 504 may be provided as a microcontroller, microprocessor, Central Processing Unit (CPU), a Graphics Processing Unit (GPU), or plurality of microprocessors that are configured to execute the instructions sets stored in memory 500. In some embodiments, the instruction sets stored in memory 500, when executed by the processor 504, may enable the device 136 to provide dental restoration functionality.

The nature of the network interface 512 may depend upon whether the network interface 512 is provided in a portable or nonportable computational device. Examples of a suitable network interface 512 include, without limitation, an Ethernet port, a USB port, an RS-232 port, an RS-485 port, a NIC, an antenna, a driver circuit, a modulator/demodulator, etc. The network interface 512 may include one or multiple different network interfaces depending upon whether the device 136 is connecting to a single network 112 or multiple different types of networks 112.

The user interface 508 may include a combination of user input devices and user output devices. For instance, the user interface 508 may include a display screen, speakers, buttons, levers, a touch-sensitive display, or any other device that is capable of enabling a service provider to interact with the device 136. The user interface 508 may also include one or more drivers for the various hardware components (such as the scanning device) that enable the service provider to interact with the device 136 or with output devices, such as the fabrication device.

The memory 500 may include one or multiple computer memory devices or computer readable media that are volatile or non-volatile. The memory 500 may include volatile and/or non-volatile memory devices. Non-limiting examples of memory 500 include Random Access Memory (RAM), Read Only Memory (ROM), flash memory, Electronically-Erasable Programmable ROM (EEPROM), Dynamic RAM (DRAM), etc.

The memory 500 may be configured to store the instruction sets depicted in addition to temporarily storing data for the processor 504 to execute various types of routines or functions. The instruction sets can enable user interaction with the device 136 or peripheral devices, such as the scanning device or fabrication device. Examples of instruction sets or data that may be stored in the memory 500 include fixture scan data 516, attachment member scan data 520, oral scan data 524, restoration design instructions 528, and communication instructions (not shown).

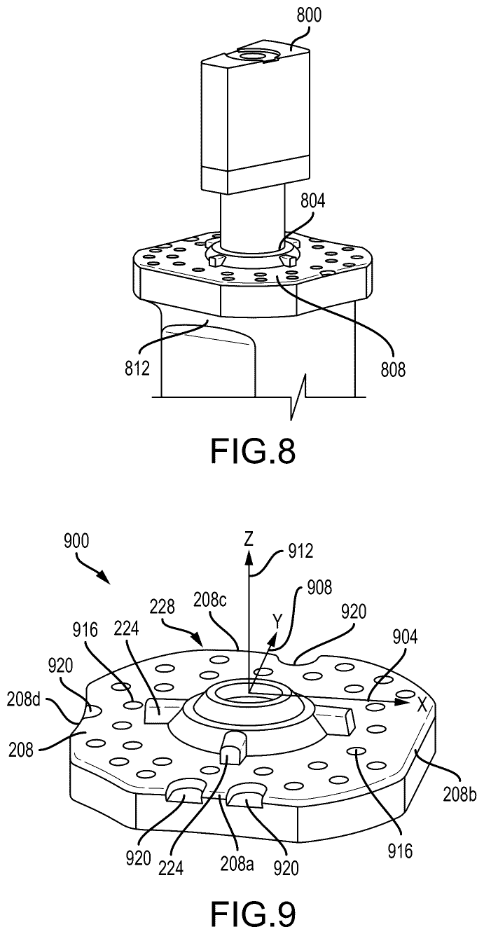

The fixture scan data includes scan data of the scan fixture 200 alone or scan fixture with the scan fixture calibration object 400 attached to the scan fixture 200. By way of illustration, FIG. 8 depicts fixture scan data comprising captured images of the scan fixture calibration object 800 attached to the analog 804 on the connection interface 808 of the scan fixture 812.

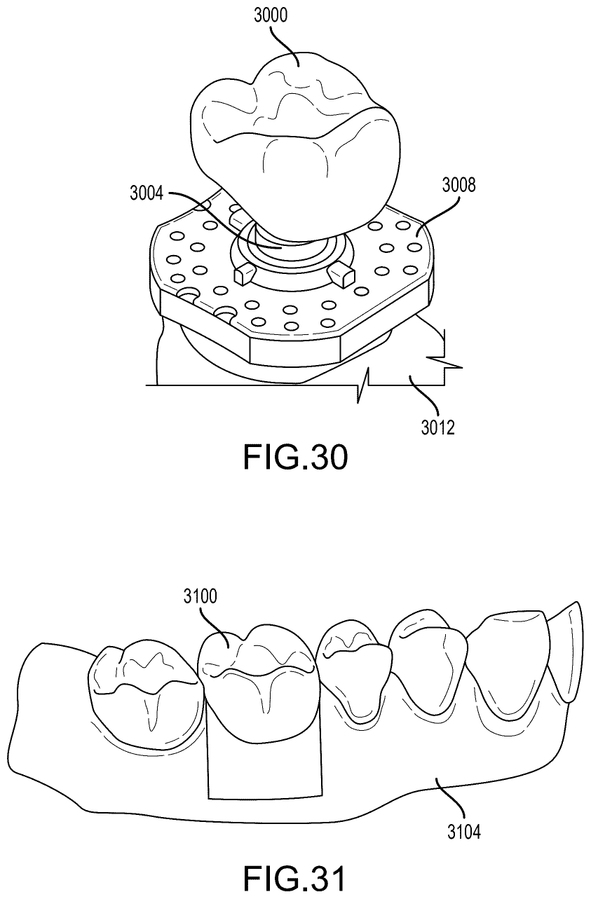

The attachment member scan data 520 includes can data of the attachment member 1200 attached to the scan fixture 200. By way of illustration, FIG. 30 depicts attachment member scan data comprising captured images of the attachment member 3000 attached to the analog 3004 on the connection interface 3008 of the scan fixture 3012.

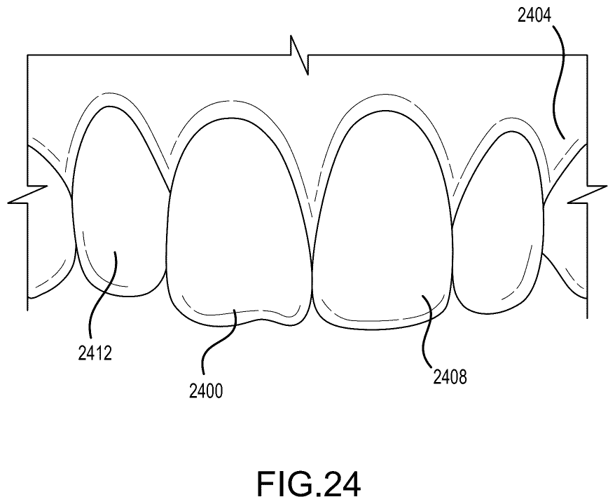

Oral scan data 524 includes scan data of the attachment member installed in the oral cavity of the patient. By way of illustration, FIG. 24 depicts oral scan data comprising captured images of the attachment member 2400 attached to an implant (not shown) in the oral cavity 2404 of the patient. The aesthetic match between the attachment member 2400 to the adjacent teeth 2408 and 2412 of the patient is visible from FIG. 24.

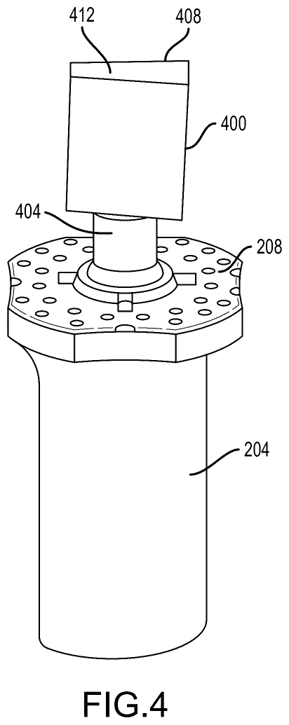

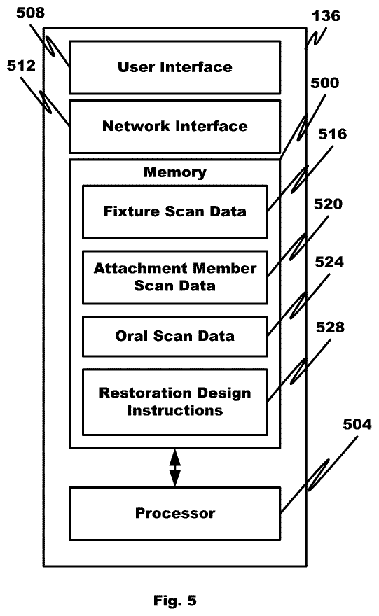

While the scan fixture and attachment member scan data shown in the figures is shown in a two dimensional view, it is to be understood that the scan data contains time and location-stamped images taken around the periphery of the scanned objects and from different vertical positions as discussed above in connection with FIGS. 3-5.

In some embodiments, the restoration design instructions 528, when executed by the processor 504, may enable the device 136 to convert or encode scan data and communicate the converted or encoded scan data to the dental restoration modeling system 104, receive and print by the fabrication device scan fixture design instructions from the system 104, and receive from the service provider 116 and provide to the restorative modeling instructions of the system 104 and vice versa dental restoration information, such as design input for abutments and prosthetics.

The communication instructions, when executed by the processor 504, may enable the device 136 to communicate with the system 104, the scanning device 120, or the fabrication device 132, or the like. In some embodiments, the communication instructions may include instructions that enable the device 136 to pair with the scanning or peripheral device and establish a communication channel with the device via the pairing. As an example, the communication instructions may include instructions that enable NFC, Bluetooth®, Wi-Fi, or other types of communication protocols. In some embodiments, the communication instructions may be configured to operate or drive the network interface 512 to facilitate direct or indirect communications with the system 104.

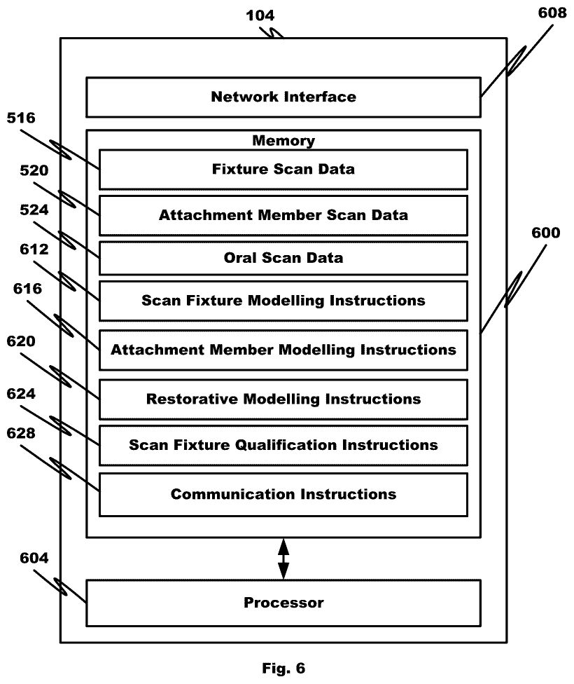

Referring to FIG. 6, an embodiment of the dental restoration modeling system 104 will be described. The dental restoration modeling system 104 is shown to include a processor 604, memory 600, and a network interface 608. In some embodiments, the processor 604 may correspond to one or many microprocessors, CPUs, microcontrollers, Integrated Circuit (IC) chips, or the like. For instance, the processor 604 may be provided as silicon, as a Field Programmable Gate Array (FPGA), an Application-Specific Integrated Circuit (ASIC), any other type of Integrated Circuit (IC) chip, a collection of IC chips, or the like. As a more specific example, the processor 604 may be provided as a microcontroller, microprocessor, Central Processing Unit (CPU), a Graphics Processing Unit (GPU), or plurality of microprocessors that are configured to execute the instructions sets stored in memory 600. In some embodiments, the instruction sets stored in memory 600, when executed by the processor 604, may enable the system 104 to provide dental restoration functionality.

The nature of the network interface 608 may depend upon whether the network interface 608 is provided in a portable or nonportable computational device. Examples of a suitable network interface 608 include, without limitation, an Ethernet port, a USB port, an RS-232 port, an RS-485 port, a NIC, an antenna, a driver circuit, a modulator/demodulator, etc. The network interface 608 may include one or multiple different network interfaces depending upon whether the system 104 is connecting to a single network 112 or multiple different types of networks 112.

The memory 600 may include one or multiple computer memory devices or computer readable media that are volatile or non-volatile. The memory 600 may include volatile and/or non-volatile memory devices. Non-limiting examples of memory 500 include Random Access Memory (RAM), Read Only Memory (ROM), flash memory, Electronically-Erasable Programmable ROM (EEPROM), Dynamic RAM (DRAM), etc.

The memory 600 may be configured to store the instruction sets depicted in addition to temporarily storing data for the processor 604 to execute various types of routines or functions. The instruction sets can enable interaction of each of the dental service provider computational devices 136 with the dental restoration modeling system 104. Examples of instruction sets that may be stored in the memory 600 or data that may be stored in the memory 600 or attached modeling database 108 include fixture scan data 516 for a plurality of different dental service providers 116 and, for each dental service provider, for a plurality of different types of nonrotational structures, attachment member scan data 520 for a plurality of different dental service providers 116, oral scan data 524 for a plurality of different patients for each dental service provider 116, scan fixture 3D modeling instructions 612, attachment member 3D modeling instructions 616, restorative 3D modeling instructions 620, scan fixture qualification instructions 624, and restoration design instructions 628, and communication instructions 632.

In some embodiments, the scan fixture modeling instructions 612, when executed by the processor 604, may enable the system 104 to convert the fixture scan data 516 for scan fixtures of a plurality of different dental service providers 116 and, for each dental service provider, for a plurality of different types of nonrotational structures into a corresponding virtual 3D scan fixture models. The instructions, for example, can be CAD software, graphical imaging software, and the like configured to process the fixture scan data 516 into the corresponding virtual 3D scan fixture model. An exemplary virtual 3D scan fixture model is shown in FIG. 9.

In some embodiments, the attachment member modeling instructions 616, when executed by the processor 604, may enable the system 104 to convert the attachment member scan data 520 for attachment members of a plurality of different dental service providers 116 and, for each dental service provider, for a plurality of different types of nonrotational structures into a corresponding virtual 3D attachment member models. The instructions, for example, can be CAD software, graphical imaging software, and the like configured to process the attachment member scan data 520 into the corresponding virtual 3D attachment member model. An exemplary virtual 3D attachment member model is shown in FIG. 14.

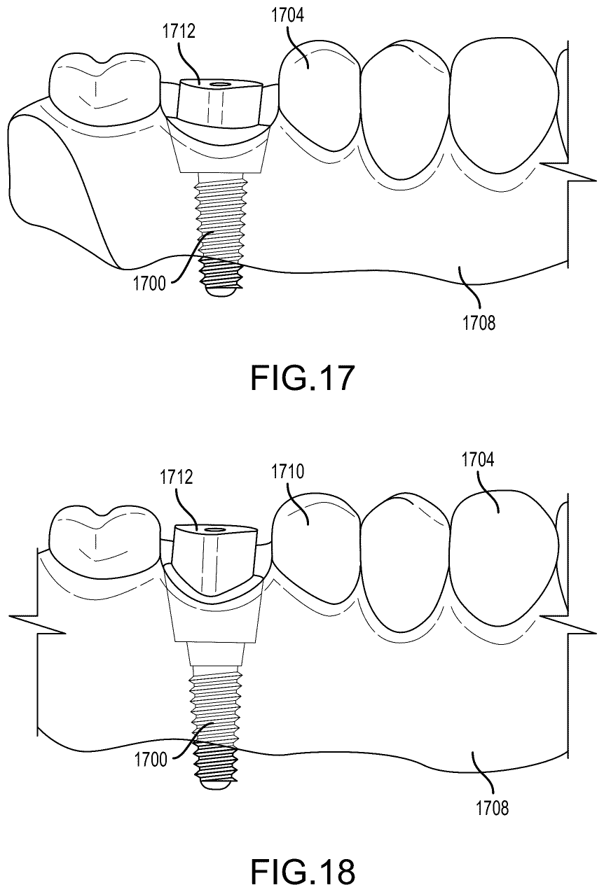

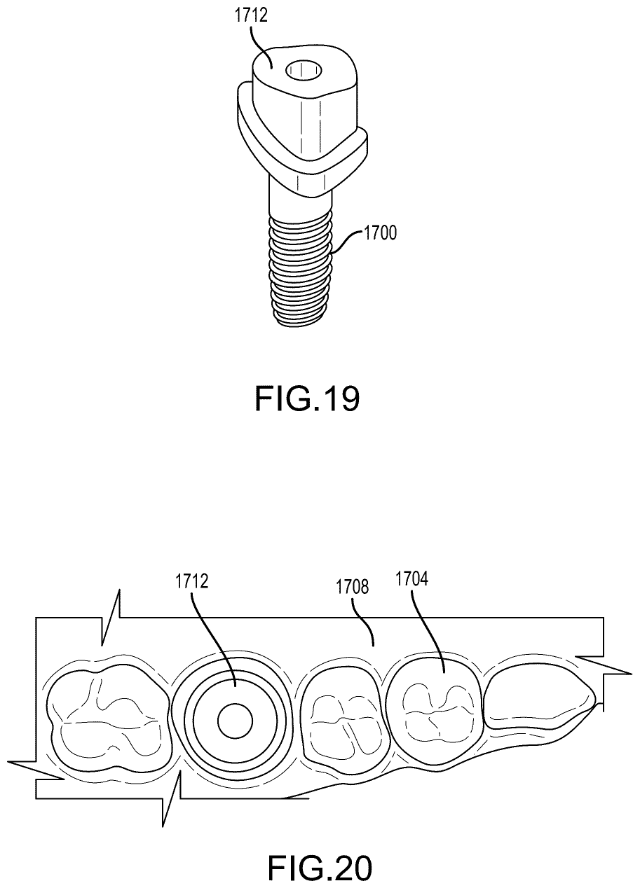

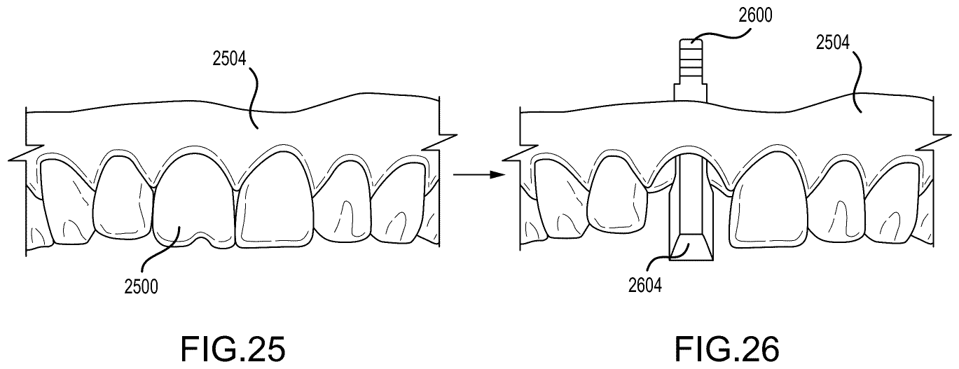

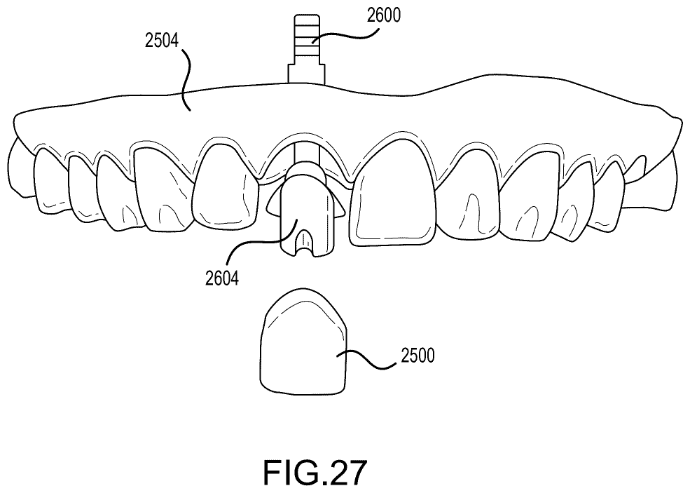

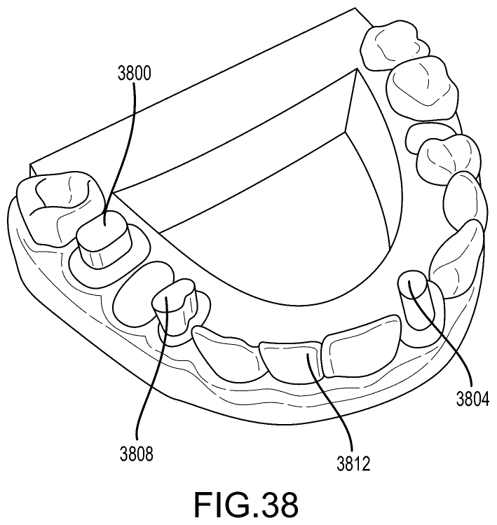

In some embodiments, the restorative modeling instructions 616, when executed by the processor 604, may enable the system 104 to convert the oral scan data 524 for a plurality of different patients for each dental service provider 116 into a corresponding virtual 3D restorative models that may include a virtual counterpart of an implant in the corresponding patient and an abutment to be attached to the implant and/or prosthetic to be attached to the abutment. The instructions, for example, can be CAD software, graphical imaging software, and the like configured to process the oral scan data 524 into the corresponding virtual 3D restorative model. An exemplary virtual 3D restorative model is shown in FIGS. 17-21, which show various displays of modeled implants and abutments that are part of a dental restoration. Referring to FIGS. 17-21 show various displays provided by a restorative 3D model of a patient's oral cavity provided by the system 104 that includes a virtual implant 1700, virtual teeth 1704, virtual gums 1708, virtual gumline 1710, and an abutment 1712. FIG. 19 shows the virtual abutment 1712 and implant 1700 in isolation from the virtual teeth and gums. Another example of a restorative 3D model is shown by the various displays of FIGS. 25-27, which show the prosthetic tooth 2500, positioned in a virtual jaw 2504 of the patient, the abutment 2504 engaged with an analog 2600 (which is representative of the implant in the patient's jaw). As will be appreciated, the restorative modeling instructions can provide instructions to a fabrication device 132 to manufacture a physical prototype (e.g., a rapid prototype model such as that depicted in FIG. 38) of the model depicted in FIGS. 25-27.

In some embodiments, the restorative modeling instructions 616, when executed by the processor 604, may enable the system 104 to operate in the registration, calibration, or qualification mode, in which the scan fixture calibration object is engaged with the scan fixture (in the absence of the attachment member) and scanned outside the patient's mouth to provide precise and accurate scanning information about the scan fixture, including conveying the position of a virtual 3D coordinate system and/or position and/or orientation of the underlying connection interface (e.g., nonrotational structure).

The communication instructions 628, when executed by the processor 604, may enable the system 104 to communicate with the device 136 of each dental service provider 116. In some embodiments, the communication instructions may be configured to operate or drive the network interface 608 to facilitate direct or indirect communications with the devices 136.

The modeling database 108-a may be configured to store one or multiple data structures that are used in connection with dental restorations. In some embodiments, the data stored in the data structure may be stored for a plurality of different service providers. As a non-limiting example, the data structure may be used to store a particular restoration service provider (e.g., case identifier), tooth undergoing restoration (e.g., tooth identifier), and/or patient (e.g., patient identifier) and an associated database library comprising fixture scan data comprising scans of the qualification and restorative scan assemblies, attachment member scan data, oral scan data, virtual 3D scan fixture model, virtual 3D attachment member model, and virtual 3D restorative model for the abutment and prosthetic to be employed in the restoration. When scanned images are received from the restoration service provider, they can be automatically paired with the database library containing previously received scanning information and models associated with the qualification and restorative scan assemblies.

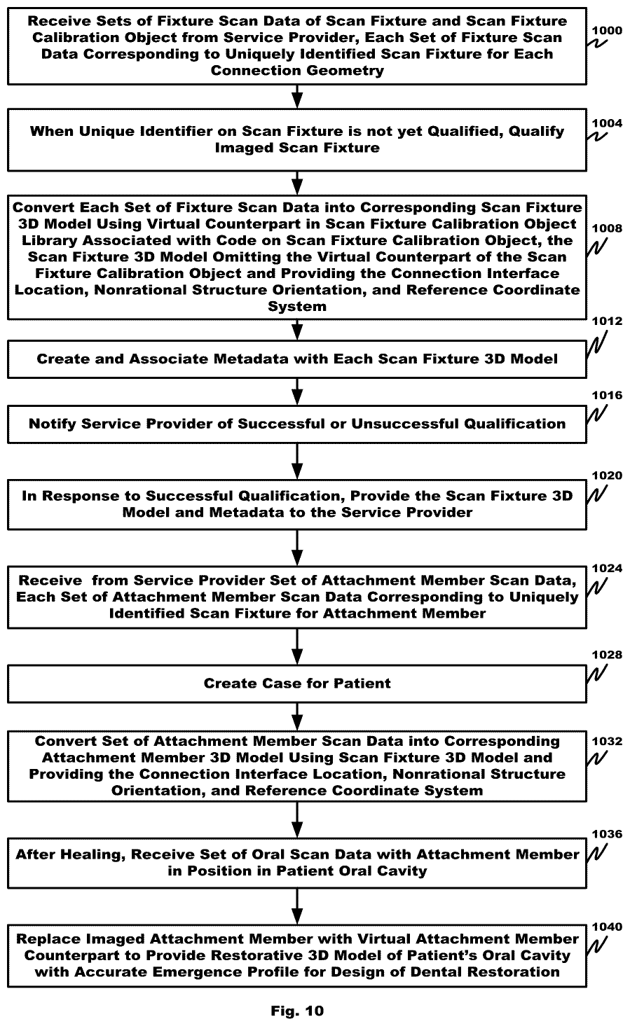

Referring now to FIG. 10, additional details of a method of performing a restoration will be described in accordance with embodiments of the present disclosure. In the following process flow descriptions, the operations may be performed in a different order than the order shown, or the operations may be performed in different orders or at different times. Certain operations may also be left out of the process flow, or other operations may be added to the process flow. It is to be understood that any device or collection of devices may perform the operations shown.

The method can begin by the dental restoration modeling system 104 receiving sets of fixture scan data of a scan fixture 200 and an attached scan fixture calibration object 400 from a service provider 116 (such as the scan data shown in FIG. 8) (step 1000). The scan fixtures can be fabricated by each service provider via the associated fabrication device 132.

The method can continue by the dental restoration modeling system 104 determining whether the unique identifier on the imaged scan fixture has been previously qualified. When the imaged scan fixture has not yet been qualified, the system 104 creates a master library associated with the scan fixture identifier. Each scan fixture having a specific type of analog or nonrotational structure submitted by a given service provider is associated with a different identifier. In other words, each identified scan fixture is persistently associated with a specific analog or nonrotational structure type. As noted, to create the master library for each identifier that comprises a virtual counterpart of the imaged scan fixture, the scan fixture scan data typically has a high-precision scan body as the scan fixture calibration object. If the scan body does not have a sufficiently high precision, the scan fixture is unable to be qualified due to an inability to determine the reference axes or datum plane shown in FIG. 9 (step 1004).

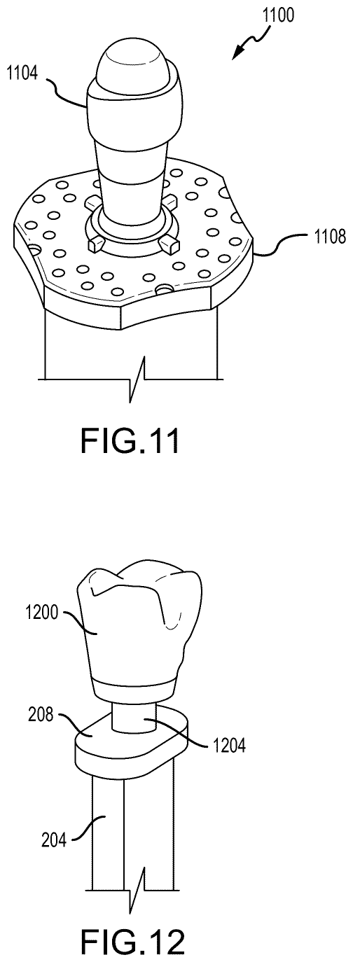

After the imaged scan fixture is qualified (as the scan data is sufficiently detailed to enable shape matching (with no more than a threshold level of mismatch) of the virtual counterpart of the scan fixture calibration object to the imaged scan fixture calibration object) or when the imaged scan fixture has previously been qualified, the system 104 can continue by converting each set of fixture scan data into a corresponding scan fixture 3D model (step 1008). This is typically done by converting the scan data into a virtual 3D model, using the virtual counterpart in scan fixture calibration object library associated with the code on the scan fixture calibration object to shape match the scan fixture calibration object to the imaged scan fixture calibration object, and positioning the reference axes and datum plane to the connection interface 208 based on the shape matched virtual counterpart. As shown in FIG. 9, the scan fixture 3D model can then omit or otherwise remove the virtual counterpart of the scan fixture calibration object from the virtual 3D model to reveal the connection interface, including the connection interface location, nonrational structure orientation of the analog, and reference coordinate system. FIG. 11 displays a scan fixture 3D model 1100 before removal of the scan fixture calibration object 1104 from the scan fixture 1108. The scan fixture 3D model is saved in the master library, in connection with the identifier on the scan fixture attachment interface, that comprises a virtual counterpart of the imaged scan fixture.

The system 104 can continue by creating and associating metadata in the master library with each scan fixture (step 1012). The metadata can include, for example, the code or identifier on the scan fixture, service provider (e.g., case identifier), tooth undergoing restoration (e.g., tooth identifier), patient (e.g., patient identifier), analog identifier, nonrotational structure description, code on the scan fixture calibration object, and timestamp.

The system 104 can continue by notifying the service provider whether the qualification was successful or unsuccessful (step 1016) and, when successfully qualified, providing the scan fixture 3D model and metadata to the service provider (step 1020).

The system 104 can continue by receiving, from the service provider, a set of attachment member scan data (generated by scanning the attachment member connected to the same scan fixture as the scan fixture calibration object), each set of attachment member scan data corresponding to the uniquely identified scan fixture attached to the attachment member (step 1024). The attachment member scan data is typically sent with a case or patient identifier (if available), associated tooth location identifier, and service provider identifier. The service provider can then place the attachment member in the patient's mouth. These operations can be performed by the service provider at the chairside while the patient is in the office.

The system 104 can continue by creating a case for the patient associated with the attachment member (step 1028). The case is typically linked to or otherwise associated with the unique identifier on the attached scan fixture.

The system 104 can continue by converting the set of attachment member scan data into a corresponding virtual 3D attachment member model based on the attachment member scan data. The scan fixture in the model is replaced by shape matching, with the virtual scan fixture in the modified 3D scan fixture model associated with the scan fixture identifier to provide a modified 3D attachment member model (step 1032). As noted, the virtual 3D scan fixture model provides the connection interface or datum plane location, nonrotational feature orientation, and reference coordinate system. The modified virtual 3D attachment member model can isolate the attachment member as shown in FIG. 14 by omitting or otherwise removing the virtual counterpart of the scan fixture.

After healing of the patient's gingiva, the system 104 can continue by receiving from the service provider a set of oral scan data with the attachment member in position in the patient's oral cavity along with an identifier of the service provider and another identifier, such as a case identifier, scan fixture identifier, or patient identifier, and tooth location identifier (when more than one tooth is being restored) to enable the appropriate virtual 3D attachment member model to be retrieved (step 1036). The oral scan data can include scans of the oral cavity with the attachment member removed to provide the gingiva profile, including the emergence profile. The patient can be sent home after the oral can data is generated by the service provider.

The system 104 can continue by converting the set of oral scan data into a corresponding virtual restorative 3D model by converting the oral scan data into a virtual 3D model of the patient's oral cavity, using the virtual counterpart of the imaged attachment member in the modified virtual 3D attachment member model associated with the identifier and tooth location identifier to shape match the virtual attachment member to the imaged attachment member, and revealing the fixture-level information (e.g., the position of the reference axes and datum plane in the patient's jaw based on the shape matched virtual counterpart) (step 1040). The restorative 3D model can then omit or otherwise remove the virtual counterpart of the attachment member from the virtual 3D model to reveal the implant seating surface, including the nonrational structure orientation of the implant, reference coordinate system, and the sub-gingival contours.

In some embodiments, an alignment check is performed in each shape matching operation noted above to confirm that the shape matching is sufficiently accurate. As will be appreciated, the attachment member is typically a provisional tooth and may change shape or crack during the healing period due to patient use. The alignment check is typically a convergence analysis to determine how closely the imaged attachment member and virtual attachment member shape match (e.g., how closely the outer contours of the imaged and virtual attachment members match). If the alignment check fails due to an unacceptable degree of shape mismatch or error between the imaged and virtual attachment members (e.g., more than 25 microns mismatch), the service provide is notified and requested to rescan the attachment member on the scan fixture to enable the virtual attachment member to be replaced by an updated virtual attachment member 3D model generated from the rescanned attachment member (step 1032).

The alignment check can be done on the upper and/or lower surface contours of the attachment member. In some applications, the alignment check is performed based only on the shape mismatch between the visible supragingival lower portions of the imaged and virtual attachment members but not on the upper supra-gingival occlusal portions (the surfaces of a tooth that come into contact with an opposing tooth when the jaw is closed). Omitting such portions from the shape matching alignment check can be done by “painting out” or obscuring such portions of the tooth from consideration in the shape matching operation. This can reduce the frequency of alignment check failure as the most likely contours to change or deform through patent usage are the exposed upper contours used in the grinding, chewing, or biting dental functions.

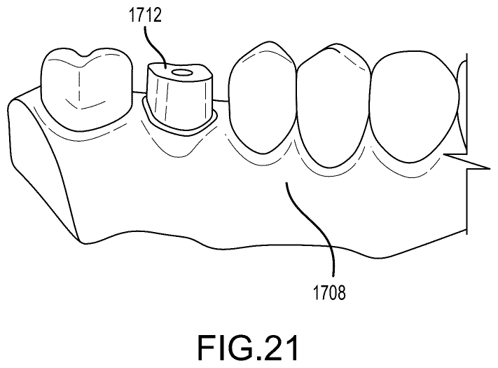

To genericize the data for compatibility with any open architecture dental design software, the abutment can be designed as shown in FIGS. 17-21, including the gingival contours, which are then “snapped into” the virtual 3D restoration model developed by the step 1040. Stated differently, the virtual abutment 3D model of FIG. 19 can be merged with the virtual restorative 3D model of step 1040 (shown in FIG. 20) to create a monolithic tissue-level virtual abutment 3D model of FIG. 21. This can be done through the shape matching operation described above in above. In other words, a virtual attachment member is designed in CAD software and, after conversion of the oral scan data into a virtual 3D model, the virtual attachment by shape matching against all or a portion (as noted above) of the virtual counterpart of the imaged attachment member in the oral scan data is substituted for the attachment member in the oral scan data and represented in the virtual 3D model to provide the restorative 3D model described above. This solution would not require steps 1000-1032 but may still use the alignment check noted above. The virtual abutment and model files can be provided to the service provider for fabrication (e.g., milling and rapid prototyping).

In some embodiments, a blended approach may be employed. A first shape match operation is performed on the supragingival outer contours of the entire imaged object (e.g., attachment member) (including the occlusal surface of the attachment member). In the event that the alignment check fails, a second shape match operation is performed on the supragingival outer contours of the attachment member (omitting the occlusal surface) and a further alignment check is performed. If the further alignment check fails, the system 104 notifies the service provider of the need to submit further (second) scan data. An advantage of this tiered approach is the use of the best available scan data in each step. Using the outer supragingival contours of the entire attachment member may yield a more accurate virtual 3D model than using simply the attachment member's non-occlusal supragingival contours.

To illustrate the principles of the disclosure, a number of examples will be discussed.

With reference to FIGS. 22-27, a first case study is illustrated. With reference to FIG. 22, the attachment member 2200 is engaged with an analog 220 in the connection interface 208 of the scan fixture 204. As shown in FIG. 23, the attachment member 2200 and attached scan fixture are positioned in the fixture receiver 304 and scanned by scanning device 120 as the fixture receiver is rotated and moved upwardly and downwardly over the rotational circumference to provide attachment member scan data. The case identifier and scan data are sent to the system 104. An instant alignment check is performed and the converted or decrypted file (e.g., a standard template or STL file) with the sub-gingival contours, preparation line, and implant coordinates is transmitted to the device 136. The system 104 and device 136 interactively design the abutment and prosthetic (e.g., permanent tooth) of FIGS. 25-27. The restorative design can proceed more efficiently since no library and/or decryption is required and can be clinically superior to what can be achieved with conventional methods since the design will precisely match and therefore support the patient's tissue contours.

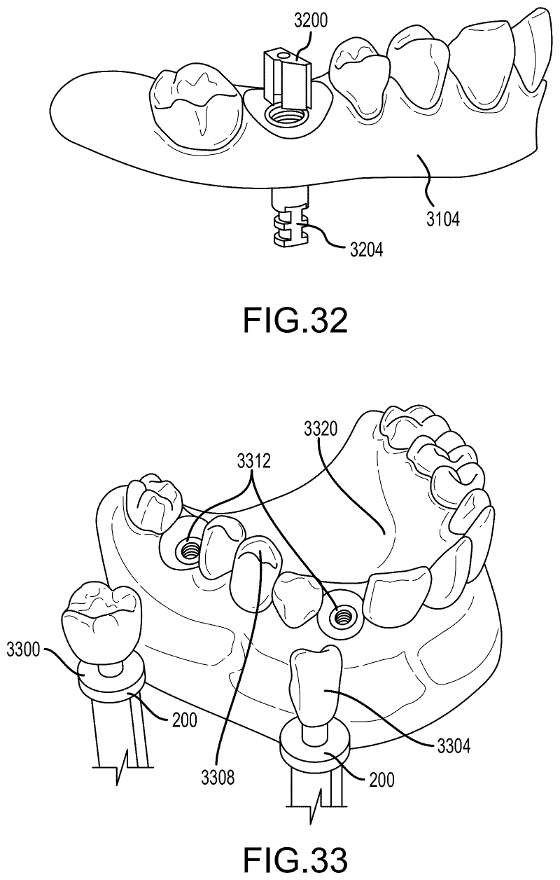

With reference to FIGS. 28-31, a second case study is illustrated in which the restoration is to install a patient-specific crown. After the attachment member 1200 is created and before the patient is discharged, the attachment member 1200 is attached (e.g., screwed into) the analog 220 of the connection interface 208 (FIG. 28). Scanning is automated and the scan fixture and attachment member 1200 are moved from top to bottom and rotated around its own axis (FIG. 29). The attachment member scan data (shown in FIG. 30) is sent to the system 104 from the scanning application (e.g., iTero™ as an example), and the system 104 creates a case for the patient. Months later after the implant has osseointegrated and the emergence profile has formed and without removing the attachment member, the oral scan data is generated and sent directly by the scanning application to the system, which creates a virtual restorative 3D model (shown in FIG. 31). In some applications, the converter in the restorative modeling instructions replaces the virtual attachment member 2100 with the fixture calibration object or other scan body 3200 and creates a perfect emergence profile in the virtual restorative 3D model (FIG. 32). The virtual restorative 3D models of FIGS. 31-32 are shown to include a virtual counterpart 3104 of the patient's jaw and an analog 3204 having the location and orientation of the implant in the patient's jaw.

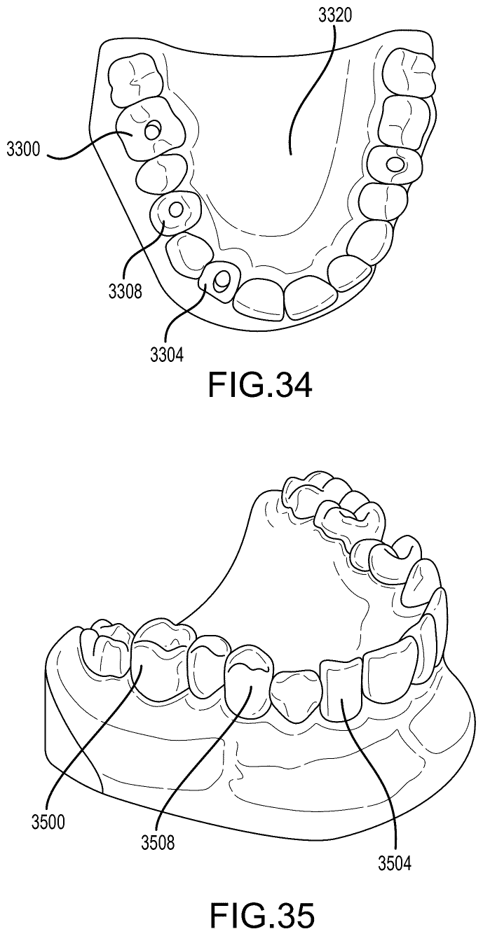

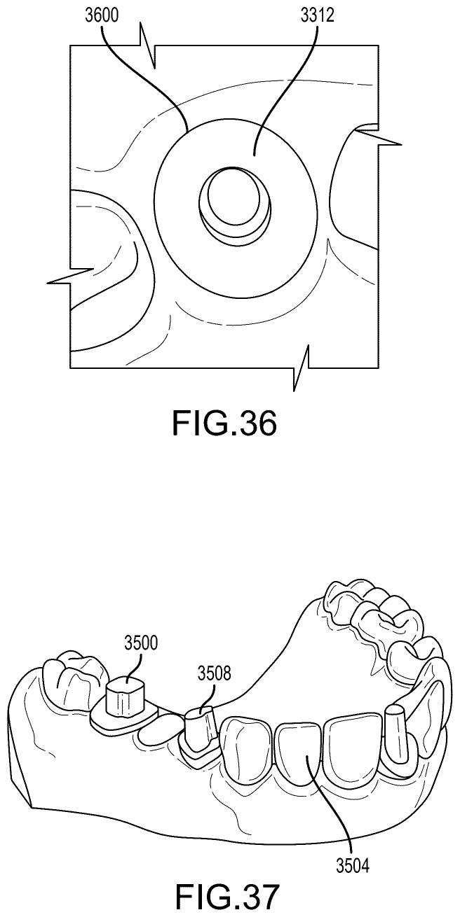

With reference to FIGS. 33-38, a third case study is illustrated in which the restoration is to install a patient-specific crown. With reference to FIGS. 33-34, first, second, and third attachment members 3300, 3304 and 3308 are sequentially engaged with a scan fixture 200 and scanned because the attachment members have the same nonrotational structure and, after scanning, engaged with implants 3312 in the patient's jaw 3320. The oral scan data is generated and converted into a virtual restorative 3D model with virtual attachment member counterparts 3500, 3508, and 3504 for the attachment members 3300, 3308, and 3304, respectively (FIGS. 35-37). FIG. 36 shows the emergence profile 3600 of the patient's jaw for one of the implant locations. FIG. 38 depicts a rapid prototype model 3812 fabricated by a service provider based on the virtual restorative 3D model, with the provisionals 3800, 3808, and 3804 corresponding to the virtual attachment members 3500, 3508, and 3504.

In another test case, a bridge was designed and fabricated using the workflow of FIG. 10.

Any of the steps, functions, and operations discussed herein can be performed continuously and automatically.

The exemplary systems and methods of this disclosure have been described in relation to dental restoration digital workflows. However, to avoid unnecessarily obscuring the present disclosure, the preceding description omits a number of known structures and devices. This omission is not to be construed as a limitation of the scopes of the claims. Specific details are set forth to provide an understanding of the present disclosure. It should however be appreciated that the present disclosure may be practiced in a variety of ways beyond the specific detail set forth herein.

Furthermore, while the exemplary aspects, embodiments, and/or configurations illustrated herein show the various components of the system collocated, certain components of the system can be located remotely, at distant portions of a distributed network, such as a LAN and/or the Internet, or within a dedicated system. Thus, it should be appreciated, that the components of the system can be combined in to one or more devices, such as a server, or collocated on a particular node of a distributed network, such as an analog and/or digital telecommunications network, a packet-switch network, or a circuit-switched network. It will be appreciated from the preceding description, and for reasons of computational efficiency, that the components of the system can be arranged at any location within a distributed network of components without affecting the operation of the system. For example, the various components can be located in a switch such as a PBX and media server, gateway, in one or more communications devices, at one or more users' premises, or some combination thereof. Similarly, one or more functional portions of the system could be distributed between a telecommunications device(s) and an associated computing device.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

Also, while the flowcharts have been discussed and illustrated in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various aspects, embodiments, and/or configurations, includes components, methods, processes, systems and/or apparatus substantially as depicted and described herein, including various aspects, embodiments, configurations embodiments, subcombinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and/or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A qualification scan assembly comprising:
an attachment surface comprising a bore; and
a structure positioned in the bore and configured to engage a scan fixture calibration object, wherein the attachment surface comprises a first identification code associated with a dental service provider or dental patient and the scan fixture calibration object comprises a second identification code associated with a virtual counterpart of the scan fixture calibration object.

2. The qualification scan assembly of claim 1, wherein the structure comprises a nonrotational structure, wherein a dental analog comprising the nonrotational structure is positioned in the bore and wherein the scan fixture calibration object comprises a scan body and wherein the first and second identification codes are different.

3. The qualification scan assembly of claim 1, wherein the attachment surface comprises one or more scannable features to orient one or both of a reference coordinate system and nonrotation structure.

4. The qualification scan assembly of claim 1, further comprising a body engaging the attachment surface, the body configured to engage a scanning device that rotates and/or translates the qualification scan assembly during scanning.

5. An attachment member scan assembly comprising:
an attachment surface comprising a bore; and
a structure positioned in the bore and configured to engage an attachment member, the attachment member being a patient-specific temporary prosthesis or abutment configured to engage an implant in an oral cavity of the patient.

6. The scan assembly of claim 5, wherein the structure comprises a nonrotational structure and wherein the attachment surface comprises a first identification code associated with a dental service provider or dental patient.

7. The scan assembly of claim 5, wherein the attachment surface comprises one or more scannable features to orient one or both of a reference coordinate system and nonrotation structure.

8. The scan assembly of claim 5, further comprising a body engaging the attachment surface, the body configured to engage a scanning device that rotates and/or translates the scan assembly during scanning.

9. A computational system comprising a processor and computer readable medium comprising instructions that, when executed, cause the processor to:
receive fixture scan data comprising an image of a scan fixture engaging a scan fixture calibration object and a dental analog having a nonrotational structure orientation;
generate, from at least a portion of the fixture scan data, a virtual three-dimensional model of the scan fixture and scan fixture calibration object; and
replace, in the virtual attachment member three-dimensional model, the image of the scan fixture with a virtual counterpart of the scan fixture calibration object to form a modified virtual three-dimensional fixture model comprising one or more of a reference coordinate system position relative to a surface of the scan fixture and an orientation of the nonrotational structure relative to a surface of the scan fixture.

10. The computational system of claim 9, wherein the instructions, when executed, cause the processor to:
receive attachment member scan data comprising an image of the scan fixture engaging an attachment member and the dental analog having the nonrotational structure orientation;
generate, from the attachment member scan data, a virtual three-dimensional attachment member model of the scan fixture and attachment member; and
replace, in the virtual three-dimensional attachment member model, the image of the scan fixture with a virtual counterpart of the scan fixture in the modified virtual three-dimensional fixture model to form a modified virtual three-dimensional attachment member model comprising one or more of a reference coordinate system position relative to a surface of the attachment member and the orientation of the nonrotational structure relative to a surface of the attachment member.

11. The computational system of claim 10, wherein the instructions, when executed, cause the processor to:
receive oral scan data of a patient's oral cavity comprising an image of at least a portion of the attachment member attached to a dental implant;
generate, from the oral scan data, a virtual three-dimensional restorative model of the oral cavity; and
replace, in the virtual three-dimensional restorative model, the image of the at least a portion of the attachment member with a virtual counterpart of the at least a portion of the attachment member in the modified virtual three-dimensional attachment member model to form a modified virtual three-dimensional restorative model comprising one or more of a seating surface of the attachment member on the dental implant, a reference coordinate system position relative to the seating surface and the orientation of the nonrotational structure relative to the seating surface.

12. The computational system of claim 11, wherein the instructions, when executed, cause the processor to remove the image of the at least a portion of the attachment member from the virtual three-dimensional restorative model to provide the modified virtual three-dimensional restorative model.

13. The computational system of claim 11, wherein the instructions, when executed, cause the processor to determine a degree of mismatch between an outer contour of the attachment member in the virtual three-dimensional restorative model and the modified virtual three-dimensional attachment member model and, when the degree of mismatch is within a predetermined range, determining that the virtual three-dimensional restorative model is invalid and repeating the receive and generate operations with a second set of oral scan data.

14. A computer readable medium comprising the instructions of claim 9.

15. A computational system comprising a processor and computer readable medium comprising instructions that, when executed, cause the processor to:
receive oral scan data of a patient's oral cavity comprising an image of at least a portion of an attachment member attached to a dental implant;
generate, from the oral scan data, a virtual three-dimensional restorative model of the oral cavity;
shape match a supragingival image of at least a portion of the attachment member with a supragingival virtual counterpart of the at least a portion of the attachment member, each of the supragingival image and virtual counterpart excluding an occlusal surface of the attachment member, and
replace, in the virtual three-dimensional restorative model, the attachment member with the shape matched virtual counterpart of the attachment member, each of the supragingival image and virtual counterpart excluding an occlusal surface of the attachment member, to form a modified virtual three-dimensional restorative model comprising one or more of a seating surface of the attachment member on the dental implant, a reference coordinate system position relative to the seating surface and the orientation of the nonrotational structure relative to the seating surface.

16. The computational system of claim 15, wherein the instructions, when executed, cause the processor to:
receive fixture scan data comprising an image of a scan fixture engaging a scan fixture calibration object and a dental analog having a nonrotational structure orientation;
generate, from the fixture scan data, a virtual three-dimensional model of the scan fixture and scan fixture calibration object; and
replace, in the virtual attachment member three-dimensional model, the image of the scan fixture with a virtual counterpart of the scan fixture calibration object to form a modified virtual three-dimensional fixture model comprising one or more of a reference coordinate system position relative to a surface of the scan fixture and an orientation of the nonrotational structure relative to a surface of the scan fixture.

17. The computational system of claim 16, wherein the instructions, when executed, cause the processor to:
receive attachment member scan data comprising an image of the scan fixture engaging the attachment member and the dental analog having the nonrotational structure orientation;
generate, from the attachment member scan data, a virtual three-dimensional attachment member model of the scan fixture and attachment member; and
replace, in the virtual three-dimensional attachment member model, the image of the scan fixture with a virtual counterpart of the scan fixture in the modified virtual three-dimensional fixture model to form a modified virtual three-dimensional attachment member model comprising one or more of a reference coordinate system position relative to a surface of the attachment member and the orientation of the nonrotational structure relative to a surface of the attachment member, the virtual counterpart of the attachment member being in the modified virtual three-dimensional attachment member model.

18. The computational system of claim 15, wherein the instructions, when executed, cause the processor to determine a degree of mismatch between an outer contour of the image of the attachment member in the virtual three-dimensional restorative model and the virtual counterpart and, when the degree of mismatch is within a predetermined range, determining that the virtual three-dimensional restorative model is invalid and repeating the receive and generate operations with a second set of oral scan data.

19. The computational system of claim 15, wherein the instructions, when executed, cause the processor to remove the image of the attachment member from the virtual three-dimensional restorative model to provide the modified virtual three-dimensional restorative model.

20. A computer readable medium comprising the instructions of claim 14.

21. A computer readable medium comprising plural libraries comprising a plurality of records, each record comprising an identifier of a scan fixture and virtual 3D models of the scan fixture and attachment member associated with the identifier, each library being associated with a unique identifier of a restoration service provider, patient and/or tooth.

22. The computer readable medium of claim 21, wherein each scan fixture identifier is associated with a specific type of nonrotational structure and wherein the virtual 3D models of the scan fixture and attachment member comprise a representation of the nonrotational structure.
